(19) United States
(12) Patent Application Publication
(10) Pub. No.: US 2005/0096361 A1
(43) Pub. Date: May 5, 2005

(54) Beta-amino ketones for the treatment of pain

(75) Inventors: Helmut Buschmann, Esplugues de Llobregat (ES); Claudia Puetz, Dueren (DE); Dieter Enders, Aachen (DE); Stefan Oberboersch, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/917,522

(22) Filed: Aug. 13, 2004

Related U.S. Application Data
(63) Continuation of application No. PCT/EP03/01319, filed on Feb. 11, 2003.

(30) Foreign Application Priority Data
Feb. 14, 2002 (DE) DE 102 06 403.2

(51) Int. Cl. A61K 31/44; A61K 31/325; C07C 271/18

(52) U.S. Cl. 514/357; 514/478; 514/485; 546/335; 560/24

(57) ABSTRACT

Beta-amino ketone compounds corresponding to the formula (I) 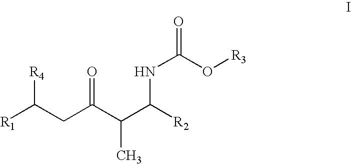 in which R1 through R4 have defined meanings, a method for producing such compounds, especially stereoselective production of such compounds, pharmaceutical compositions containing such beta-amino ketone compounds, and methods of using such compounds in the treatment of various conditions such as pain, anxiety, depression and/or epilepsy.

### CROSS REFERENCE TO RELATED APPLICATIONS

This application is a continuation of international patent application no. PCT/EP03/01319, filed Feb. 11, 2003, designating the United States of America, and published in German as WO 03/068731 A1, the entire disclosure of which is incorporated herein by reference. Priority is claimed based on Federal Republic of Germany patent application no. DE 102 06 403.2, filed Feb. 14, 2002.

### BACKGROUND OF THE INVENTION

The pesent invention relates to beta-amino ketones, to a process for the—in particular stereoselective—production thereof, to pharmaceutical compositions containing beta-amino ketones according to the invention and to the use of beta-amino ketones according to the invention for the production of pharmaceutical compositions for the treatment of pain.

The treatment of chronic and non-chronic pain has great importance in medicine. There is a worldwide need for effective methods of pain therapy for patient-friendly and purposeful treatment of chronic and non-chronic pain conditions, this being taken to mean the successful and satisfactory treatment of pain for the patient. This is documented in the large number of scientific papers that have recently appeared in the field of applied analgesics and fundamental research on nociception.

Tramadol hydrochloride-(1RS,2RS)-2-[(dimethylamino)methyl]-1-(3-methoxyphenyl)cyclohexanol hydrochloride) is a known drug for the treatment of intense pain. Aminomethyl-aryl-cyclohexanol derivatives such as tramadol ((1RS,2RS)-2-[(dimethylamino)methyl]-1-(3-methoxyphenyl)cyclohexanol hdrochloride) or alternatively hydroxylated tramadol derivatives of the type described, for example, in EP 753506 A1 can accordingly have an analgesic action, or may be used as intermediates for the production of analgesically acting substances (for example 4- or 5-substituted tramadol analogues which are described in EP 753 506 A1 or EP 780 369 A1). Tramadol has a special position among the centrally acting analgesics insofar as this active ingredient markedly inhibits pain without having the known side effects of opioids (J. Pharmacol. Exptl. Ther. 267, 331 (1993)), the enantiomers of tramadol as well as the enantiomers of tramadol metabolites participating in the analgesic action (J. Pharmacol. Exp. Ther. 260, 275 (1992)).

There remains, however, a need for further analgesically active substances.

### SUMMARY OF THE INVENTION

It is therefore an object of the invention to provide new analgesically active substances.

It is also an object of the invention to provide pharmaceutical compositions comprising new analgesically active substances.

Another object of the invention is to provide a method for preparing new analgesically active substances.

A further object of the invention is to provide new method of treating or inhibiting pain with new analgesically active substances.

The invention accordingly relates to beta-amino ketones corresponding to formula I 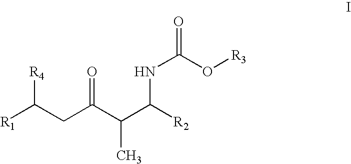
wherein
R₁ is selected from substituted or unsubstituted, branched or unbranched, saturated or unsaturated C₁₋₄ alkyl; or singly or multiply substituted or unsubstituted phenyl;
R₂ is selected from H; substituted or unsubstituted, branched or unbranched, saturated or unsaturated C₁₋₄ alkyl; respectively singly or multiply substituted or unsubstituted aryl or heteroaryl bound by a (CH₂)ₙ group (wherein n=0, 1 or 2);
R₃ is selected from substituted or unsubstituted, branched or unbranched, saturated or unsaturated C₁₋₄ alkyl; or singly or multiply substituted or unsubstituted benzyl; and
R₄ is selected from H; or substituted or unsubstituted, saturated or unsaturated C₁₋₂ alkyl;
   optionally in the form of their racemates, their pure stereoisomers, in particular enantiomers or diastereomers, or in the form of mixtures of the stereoisomers, in particular the enantiomers or diastereomers, in any mixing ratio; in the form illustrated or in the form of their acids or their bases or in the form of their salts, in particular the physiologically acceptable salts, or in the form of their solvates, in particular the hydrates.

It is particularly preferred if these beta-amino ketones according to the invention are in a stereoselectively pure form, in particular in an anti-conformation according to formula Ia or in a syn-conformation according to formula Ib 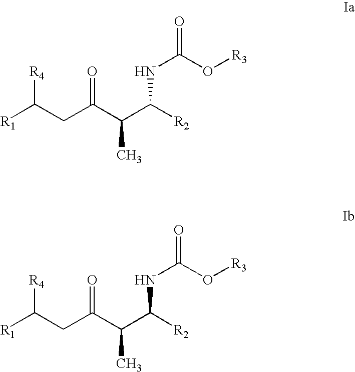
The invention further also relates to beta-amino ketones according to general formula XX 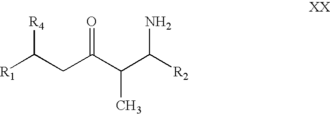
wherein
R₁ is selected from substituted or unsubstituted, branched or unbranched, saturated or unsaturated C₁₋₄ alkyl; or singly or multiply substituted or unsubstituted phenyl;
R₂ is selected from H; substituted or unsubstituted, branched or unbranched, saturated or unsaturated C₁₋₄ alkyl; respectively singly or multiply substituted or unsubstituted aryl or heteroaryl bound by a (CH₂)ₙ group (wherein n=0, 1 or 2); and
R₄ is selected from H; or substituted or unsubstituted, saturated or unsaturated C₁₋₂ alkyl;
   optionally in the form of their racemates, their pure stereoisomers, in particular enantiomers or diastereomers, or in the form of mixtures of the stereoisomers, in particular the enantiomers or diastereomers, in any mixing ratio; in the form illustrated or in the form of their acids or their bases or in the form of their salts, in particular the physiologically acceptable salts, or in the form of their solvates, in particular the hydrates.

It is particularly preferred if these beta-amino ketones according to the invention are in a stereoselectively pure form, in particular in an anti-conformation according to formula XXa or in a syn-conformation according to XXb 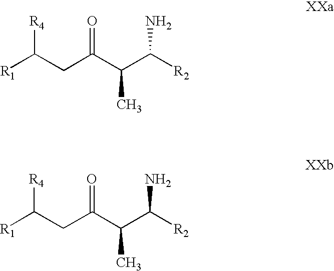
The compounds illustrated are effective analgesics.

In the context of this invention, the terms alkyl and cycloalkyl radicals refer to saturated and unsaturated (but not aromatic), branched, unbranched and cyclic hydrocarbons which may be unsubstituted or singly or multiply substituted. In this regard, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4 or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7 or C8-alkyl, C₁₋₁₀-alkyl represents C1-, C2-, C3-, C4-, C5-, C6-, C7-, C8,- C9- or C10-alkyl and C₁₋₁₈-alkyl represents C1-, C2-, C3-, C4-, C5-, C6-, C7-, C8-, C9-, C10-, C11-, C12, C13, C14-, C15-, C16-, C17- or C18-alkyl. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. With respect to cycloalkyl, the term also comprises saturated cycloalkyls, in which one or two carbon atoms are replaced by a heteroatom, S, N or O. However, the term cycloalkyl also includes singly or multiply, preferably singly, unsaturated cycloalkyls without a heteroatom in the ring, if the cycloalkyl is not an aromatic system. The alkyl and cycloalkyl radicals are preferably methyl, ethyl, vinyl (ethenyl), propyl, allyl (2-propenyl), 1-propynyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylyclopropyl, cyclopropylmethyl, cyclobutyl, cyclopentyl, cyclopentylmethyl, cyclohexyl, cycloheptyl, cyclooctyl, but also adamantyl, CHF₂, CF₃ or CH₂OH and pyrazolinone, oxopyrazolinone, [1,4]dioxane or dioxolane.

In this case, in conjunction with alkyl and cycloalkyl—unless this is not explicitly defined otherwise—the term “substituted” in the context of this invention means the replacement of at least one (optionally also more) hydrogen radical(s) by F, Cl, Br, I, NH₂, SH or OH. The terms “multiply substituted” or “substituted” with multiple substitution mean that the substitution is made both on different and on the same atoms multiply with the same or different substituents, for example threefold on the same carbon atom as in the case of CF₃ or at different points as in the case of —CH(OH)—CH═CH—CHCl₂. Particularly preferred substituents in this case are F, Cl and OH. With respect to cycloalkyl, the hydrogen radical can also be replaced by OC₁₋₃ alkyl or C₁₋₃ alkyl (singly or multiply substituted or unsubstituted respectively), in particular methyl, ethyl, n-propyl, i-propyl, CF₃, methoxy or ethoxy.

The term (CH₂)₃₋₆ means —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—. (CH₂)₁₋₄ means —CH₂—, —CH₂—CH₂—, —CH₂—CH₂—CH₂— and —CH₂—CH₂—CH₂—CH₂. (CH₂)₄₋₅ means —CH₂—CH₂—CH₂—CH₂— and —CH₂—CH₂—CH₂—CH₂—CH₂—, etc.

The term “aryl” radical refers to ring systems with at least one aromatic ring but without heteroatoms in a single one of the rings. Examples include phenyl, naphthyl, fluoranthenyl, fluorenyl, tetralinyl or indanyl, in particular 9H fluorenyl or anthracenyl radicals which can be unsubstituted or singly or multiply substituted.

The term “heteroaryl” radical refers to heterocyclic ring systems with at least one unsaturated ring which contain one or more heteroatoms selected from the group consisting of nitrogen, oxygen and sulfur and which can also be singly or multiply substituted. Examples of heteroaryl radicals include furan, benzofuran, thiophene, benzothiophene, pyrrole, pyridine, pyrimidine, pyrazine, quinoline, isoquinoline, phthalazine, benzo[1,2,5]thiadiazole, benzothiazole, indole, benzotriazole, benzodioxolan, benzodioxan, carbazole, indole and quinazoline.

Unless explicitly otherwise defined, the term “substituted” as used in conjunction with “aryl” or “heteroaryl” refers to the substitution of the aryl or heteroaryl radical by R²³, OR²³, halogen (preferably F and/or Cl), CF₃, CN, NO₂, NR²⁴R²⁵, C₁₋₆-alkyl (saturated), C₁₋₆-alkoxy, C₃₋₈-cycloalkoxy, C₃₋₈-cycloalkyl or a C₂₋₆-alkylene.

In this case the radical R²³ represents H, a C₁₋₁₀ alkyl, preferably a C₁₋₆ alkyl, an aryl or heteroaryl radical or an aryl or heteroaryl radical bound by C₁₋₃ alkyl, saturated or unsaturated, or a C₁₋₃ alkylene-group-bound aryl or heteroaryl radical, wherein these aryl or heteroaryl radicals are not themselves substituted by aryl or heteroaryl radicals.

The radicals R²⁴ and R²⁵, which may be the same or different, represent H, a C₁₋₁₀ alkyl, preferably a C₁₋₆ alkyl, an aryl radical, a heteroaryl radical or an aryl or heteroaryl radical bound by saturated or unsaturated C₁₋₃ alkyl or a C₁₋₃ alkylene-group-bound aryl or heteroaryl radical, wherein these aryl or heteroaryl radicals are not themselves substituted by aryl or heteroaryl radicals. Alternatively, the radicals R²⁴ and R²⁵ together mean CH₂CH₂OCH₂CH₂, CH₂CH₂NR²⁶CH₂CH₂ or (CH₂)₃₋₆.

The radical R₂₆ represents H, a C₁₋₁₀ alkyl, preferably a C₁₋₆ alkyl, an aryl radical, a heteroaryl radical or an aryl or heteroaryl radical bound by saturated or unsaturated C₁₋₃ alkyl or a C₁₋₃ alkylene-group-bound aryl or heteroaryl radical, wherein these aryl or heteroaryl radicals are not themselves substituted by aryl or heteroaryl radicals.

The term “salt” refers to any form of the active ingredient according to the invention in which it assumes a charged or ionic form and is associated with a counter ion (a cation or anion) or is in solution. This also includes complexes of the active ingredient with other molecules and ions, in particular complexes complexed by ionic interactions. In particular, physiologically acceptable salts are taken to mean cations or bases and physiologically acceptable salts with anions or acids.

In the context of the invention, the term “physiologically acceptable salts with cations or bases” refers to salts of at least one of the compounds according to the invention—usually a (deprotonated) acid—as an anion with at least one, preferably inorganic, cation, which are physiologically acceptable, in particular when applied to humans and/or mammals. The salts of the alkaline and alkaline earth metals are preferred, but also salts with NH₄⁺. Mono- or di-sodium, mono- or di-potassium, magnesium or calcium salts are particularly preferred.

In the context of the invention, the term “physiologically acceptable salt with anions or acids” refers to salts of at least one of the compounds according to the invention—usually protonated, for example on nitrogen—as a cation with at least one anion which are physiologically acceptable—in particular when applied to humans and/or mammals. In the context of this invention this is taken to mean, in particular, the salt formed with a physiologically acceptable acid, namely salts of the respective active ingredient with inorganic or organic acids, which are physiologically acceptable—in particular when applied to humans and/or mammals. Examples of physiologically acceptable salts of certain acids include salts of: hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, 1,1-dioxo-1,2,dihydro-1λ⁶-benzo[d]isothiazol-3-one (saccharic acid), monomethyl sebacic acid, 5-oxo-proline, hexane-1-sulfonic acid, nicotinic acid, 2-, 3- or 4-amino benzoic acid, 2,4,6-trimethyl-benzoic acid, α-lipoic acid, acetyl glycine, acetyl salicylic acid, hippuric acid and/or aspartic acid. Hydrochloride salt is particularly preferred.

In particularly preferred beta-amino ketones according to the invention, R₁ is selected from methyl, ethyl, iso-propyl or phenyl substituted by R₅ in the para position, in particular from methyl, ethyl, iso-propyl; or wherein R₅ is selected from OC₁₋₄ alkyl or C₁₋₄ alkyl (optionally substituted or unsubstituted, branched or unbranched, saturated or unsaturated) or halogen or phenyl substituted in the para position.

It is particularly preferred for the beta-amino ketones according to the invention if R₄ is selected from H or CH₃, in particular H.

The invention further relates to beta-amino ketones corresponding to formula II 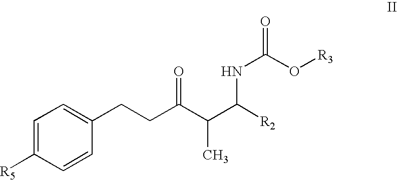
wherein
R₂ is selected from H; substituted or unsubstituted, branched or unbranched, saturated or unsaturated C₁₋₄ alkyl; respectively singly or multiply substituted or unsubstituted aryl or heteroaryl bound by a (CH₂)ₙ group (wherein n=0, 1 or 2).
R₃ is selected from substituted or unsubstituted, branched or unbranched, saturated or unsaturated C₁₋₄ alkyl; or singly or multiply substituted or unsubstituted benzyl; and
R₅ is selected from OC₁₋₄ alkyl or C₁₋₄ alkyl (respectively substituted or unsubstituted, branched or unbranched, saturated or unsaturated) or halogen,
   optionally in the form of their racemates, their pure stereoisomers, in particular enantiomers or diastereomers, or in the form of mixtures of the stereoisomers, in particular the enantiomers or diastereomers, in any mixing ratio; in the form illustrated or in the form of their acids or their bases or in the form of their salts, in particular the physiologically acceptable salts, or in the form of their solvates, in particular the hydrates.

It is particularly preferred if the beta-amino ketones according to the invention are in a stereoselectively pure form, in particular in an anti-conformation according to formula IIa or in a syn-conformation according to formula IIb

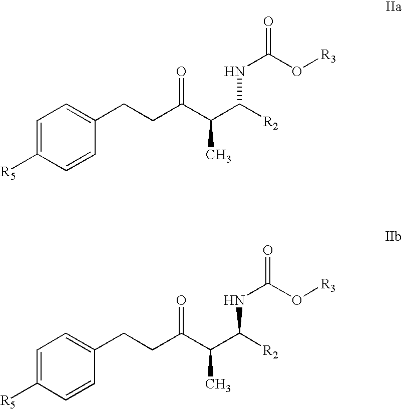

The invention further relates to beta-amino ketones corresponding to formula XXI 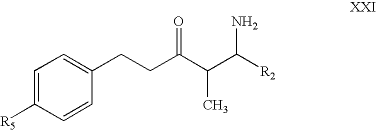
wherein
R₂ is selected from H; substituted or unsubstituted, branched or unbranched, saturated or unsaturated C₁₋₄ alkyl; respectively singly or multiply substituted or unsubstituted aryl or heteroaryl bound by a (CH₂)ₙ group (wherein n=0, 1 or 2); and
R₅ is selected from OC₁₋₄ alkyl or C₁₋₄ alkyl (respectively substituted or unsubstituted, branched or unbranched, saturated or unsaturated) or halogen,
   optionally in the form of their racemates, their pure stereoisomers, in particular enantiomers or diastereomers, or in the form of mixtures of the stereoisomers, in particular the enantiomers or diastereomers, in any mixing ratio; in the form illustrated or in the form of their acids or their bases or in the form of their salts, in particular the physiologically acceptable salts, or in the form of their solvates, in particular the hydrates.

It is particularly preferred if these beta-amino ketones according to the invention are in a stereoselectively pure form, in particular in an anti-conformation according to formula XXIa or in a syn-conformation according to formula XXIb

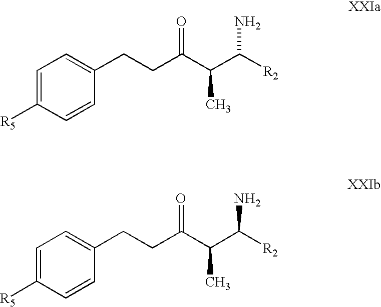

If R₅ is present in the aforementioned beta-amino ketones according to the invention, it is particularly preferred if R₅ is selected from fluorine, chlorine, bromine, iodine, methyl, ethyl, iso-propyl, iso-butyl, tert.-butyl, methoxy and ethoxy, in particular from bromine, iodine, tert.-butyl or methoxy, most preferably bromine.

It is particularly preferred for the aforementioned beta-amino ketones according to the invention if R₂ is selected from respectively singly or multiply substituted or unsubstituted aryl or heteroaryl, in particular from respectively singly or multiply substituted or unsubstituted phenyl, furyl, thiophenyl or pyridine, especially preferably from unsubstituted phenyl, furyl, thiophenyl or pyridine or from phenyl substituted by R₆ in the para-position, wherein R₆ is selected from OC₁₋₄ alkyl, C₁₋₄ alkyl or halogen; in particular methoxy, ethoxy, OCF₃ methyl, ethyl, tert.-butyl, i-propyl, CF₃ F, Cl, Br and I, especially preferably methoxy and methyl.

It is also particularly preferred for the aforementioned beta-amino ketones according to the invention if R₂ is selected from H; substituted or unsubstituted, branched or unbranched, saturated or unsaturated C₃₋₄alkyl; or respectively singly or multiply substituted or unsubstituted benzyl, phenyl, pyridyl, thiophenyl or furyl, in particular from H; unsubstituted iso-propyl; unsubstituted phenyl, furyl, thiophenyl or pyridyl or from phenyl substituted by R₆ in the para-position, wherein R₆ is selected from OC₁₋₄ alkyl, C₁₋₄ alkyl or halogen; in particular methoxy, ethoxy, OCF₃ methyl, ethyl, tert.-butyl, i-propyl, CF₃ F, Cl, Br I; especially preferably methoxy and methyl.

If R₃ is present in the aforementioned beta-amino ketones according to the invention, it is particularly preferred if R₃ is selected from C₂H₅, CH₃, i-propyl, tert.-propyl; or singly or multiply substituted or unsubstituted benzyl, in particular tert.-butyl or unsubstituted benzyl, especially preferably tert.-butyl.

The beta-amino ketones according to the invention are particularly preferably selected from the group consisting of:
[5-(4-tert-butyl-phenyl)-1-isopropyl-2-methyl-3-oxo-pentyl]carbamic acid benzylester,
[1-benzyl-5-(4-bromo-phenyl)-2-methyl-3-oxo-pentyl]carbamic acid benzylester,
[5-(4-bromo-phenyl)-2-methyl-3-oxo-1-phenyl-pentyl]-carbamic acid tert-butylester,
[5-(4-bromo-phenyl)-1-furan-2-yl-2-methyl-3-oxo-pentyl]-carbamic acid tert-butylester,
[5-(4-bromo-phenyl)-1-(4-tert-butyl-phenyl)-2-methyl-3-oxo-pentyl]-carbamic acid tert-butylester,
[5-(4-bromo-phenyl)-1-(4-methoxy-phenyl)-2-methyl-3-oxo-pentyl]-carbamic acid tert-butylester,
[5-(4-bromo-phenyl)-2-methyl-3-oxo-1-p-tolyl-pentyl]-carbamic acid tert-butylester,
[5-(4-bromo-phenyl)-2-methyl-3-oxo-pentyl]-carbamic acid tert-butylester,
[5-(4-tert-butyl-phenyl)-1-furan-2-yl-2-methyl-3-oxo-pentyl]-carbamic acid tert-butylester,
[5-(4-methoxy-phenyl)-2-methyl-3-oxo-1-phenyl-pentyl]-carbamic acid tert-butylester,
(2-methyl-3-oxo-1-phenyl-heptyl)-carbamic acid tert-butylester
[5-(4-tert-butyl-phenyl)-2-methyl-3-oxo-1-phenyl-pentyl]-carbamic acid tert-butyl ester.
[5-(4-methoxyphenyl)-2-methyl-3-oxo-1-(4-trifluoromethylphenyl)pentyl]-carbamic acid tert-butylester
   optionally in the form of their racemates, their pure stereoisomers, in particular enantiomers or diastereomers, or in the form of mixtures of the stereoisomers, in particular the enantiomers or diastereomers, in any mixing ratio; in the form illustrated or in the form of their acids or their bases or in the form of their salts, in particular the physiologically acceptable salts, or in the form of their solvates, in particular the hydrates.

Pure stereoisomeric compounds are becoming increasingly important in the pharmaceutical industry. They can exert quite different effects. A particular core of this invention is therefore a process by which exactly this, i.e. high stereoselective synthesis, can be achieved. However, the processes may obviously also be employed non-selectively for the production of the compounds according to the invention.

The invention further relates to a process for producing beta-amino ketones corresponding to formula I according to the invention as shown in reaction diagram III: 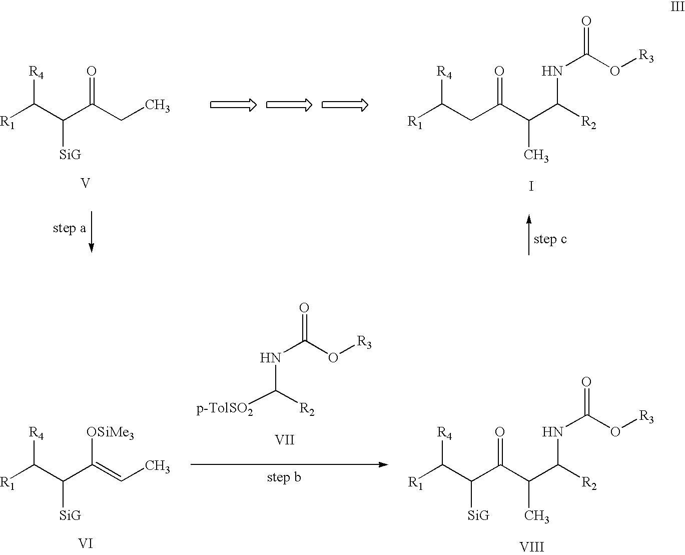
wherein GSi=tert.-hexyl-(CH₂)₂Si,
characterized in that:
in step a:
   the temperature is kept at <−70° C.,
   a non-polar organic solvent is used,
   TMSCI is used, and
   the reaction takes place in the presence of a base;
in step b:
   the temperature is kept at <0° C.,
   a polar or non-polar organic solvent is used, and
   the reaction takes place in the presence of a Lewis acid;
in step c:
   the temperature is kept at <0° C.,
   a non-polar organic solvent is used, and
   TBAF or HF is used.

The invention further relates to a process for producing beta-amino ketones according to the invention corresponding to formula II, as shown in reaction diagram IIIa: 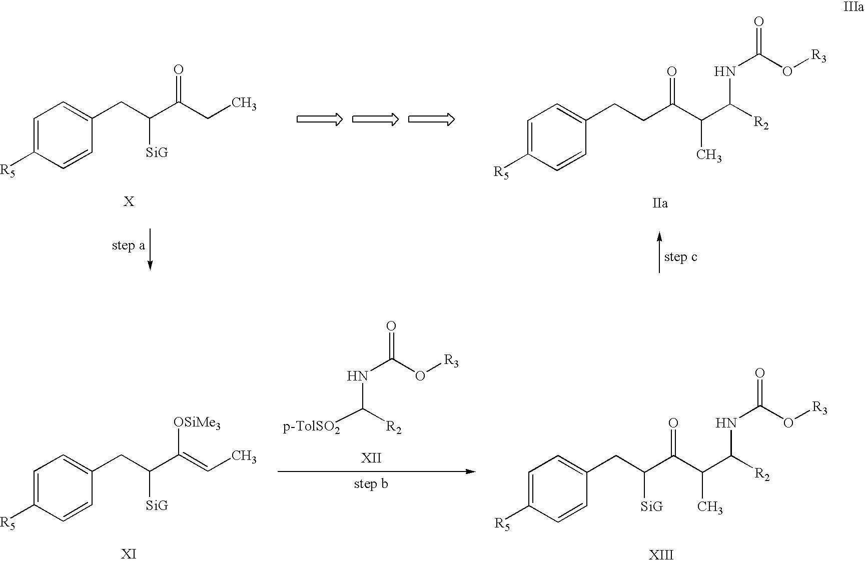
wherein GSi=tert.-hexyl-(CH₂)₂Si,
characterized in that
in step a:
   the temperature is kept at <−70° C.,
   a non-polar organic solvent is used,
   TMSCI is used, and
   the reaction takes place in the presence of a base;
in step b:
   the temperature is kept at <0° C.,
   a polar or non-polar organic solvent is used, and
   the reaction takes place in the presence of a Lewis acid;
in step c:
   the temperature is kept at <0° C.,
   a non-polar organic solvent is used, and
   TBAF or HF is used.

It is particularly preferred for reaction diagram III or IIIa if,
in step a:
   the temperature is kept at <−78° C., and/or
   THF is used as the non-polar organic solvent, and/or
   LDA is used as the base; and
   TMSCI is used;
      and/or
in step b:
   the temperature is kept at <−78° C., and/or
   CH₂Cl₂ is used as the polar organic solvent, and/or
   TiCl₄ or SnCl₄, preferably TiCl₄ is used as the Lewis acid; and/or
   KH is particularly preferably additionally used;
      and/or
in step c:
   the temperature is kept at <−78° C., and/or
   THF is used as the non-polar organic solvent and/or
   NH₄F is used to adjust a pH <7, and
   TBAF or HF is used.

The invention further relates to a process for the stereoselective production of anti-beta-amino ketones corresponding to formula IIa according to reaction diagram IIIb: 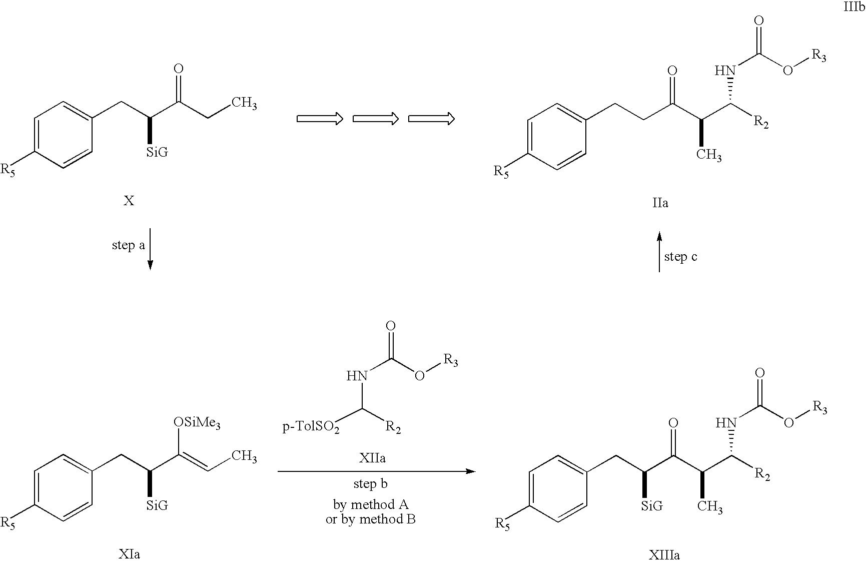
wherein GSi=tert.-hexyl-(CH₂)₂Si,
characterised in that
in step a:
   the temperature is kept at <−70° C.,
   a non-polar organic solvent is used,
   TMSCI and HMPA are used, and
   the reaction takes place in the presence of a base;
in step b:
   the temperature is kept at <−70° C.,
   a polar organic solvent is used, and
   the reaction takes place in the presence of a Lewis acid;
in step c:
   the temperature is kept at <−70° C.,
   a non-polar organic solvent is used,
   TBAF or HF is used, and
   the reaction takes place at a pH <7.

It is particularly preferred for reaction diagram IIIb if
in step a:
   the temperature is kept at <−78° C., and/or
   THF is used as the non-polar organic solvent, and/or
   LDA is used as the base; and
   TMSCI and HMPA are used;
      and/or
in step b:
   the temperature is kept <−78° C., and/or
   CH₂Cl₂ is used as the polar organic solvent, and/or
   TiCl₄ or SnCl₄, preferably TiCl_{4,} is used as the Lewis acid; and/or
   KH is particularly preferably additionally used;
      and/or
in step c:
   the temperature is kept at <−78° C., and/or
   THF is used as the non-polar organic solvent, and/or
   NH₄F is used to adjust a pH <7, and
   TBAF or HF is used.

The invention further relates to a process for producing beta-amino ketones corresponding to formula I as shown in reaction diagram IV: 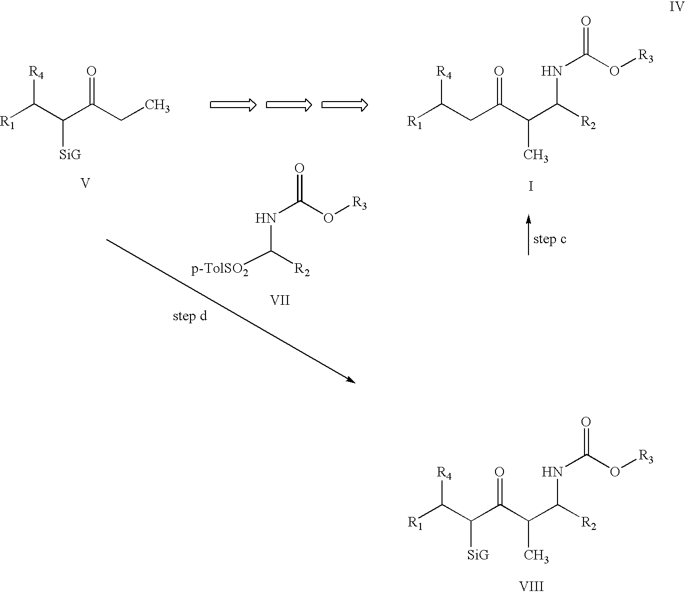
wherein GSi=tert.-hexyl-(CH₂)₂Si,
characterised in that
in step d:
   the temperature is kept at <0° C.,
   a non-polar organic solvent is used, and
   the reaction takes place in the presence of a base;
in step c:
   the temperature is kept at <0° C.,
   a non-polar organic solvent is used, and
   TBAF or HF is used.

The invention further relates to a process for the stereoselective production of syn-beta-amino ketones corresponding to formula Ib, as shown in reaction diagram IVa 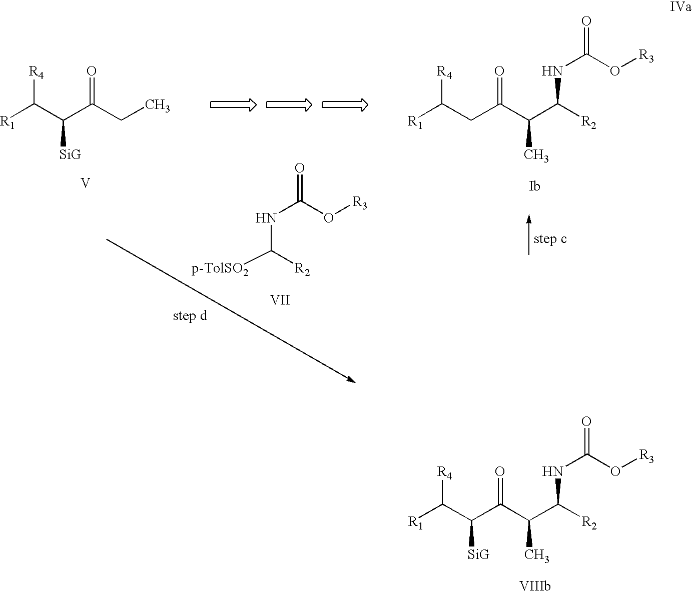
wherein GSi=tert.-hexyl-(CH₂)₂Si,
characterised in that
in step d:
   the temperature is kept at <−70° C.,
   a non-polar organic solvent is used,
   HMPA is used, and
   the reaction takes place in the presence of a base;
in step c:
   the temperature is kept at <−70° C.,
   a non-polar organic solvent is used,
   TBAF or HF is used, and
   the reaction takes place at a pH <7.

The invention further relates to a process for producing beta-amino ketones corresponding to formula II, as shown in reaction diagram IVb 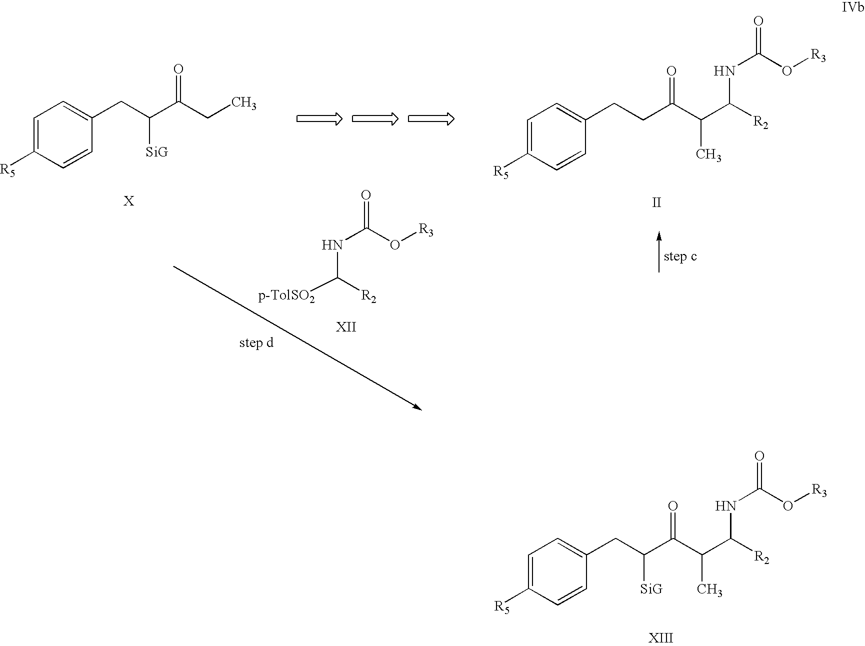
wherein GSi=tert.-hexyl-(CH₂)₂Si,
characterised in that
in step d:
   the temperature is kept at <0° C.,
   a non-polar organic solvent is used, and
   the reaction takes place in the presence of a base;
in step c:
   the temperature is kept at <0° C.,
   a non-polar organic solvent is used, and
   TBAF or HF is used.

The invention further relates to a process for the stereoselective production of anti-beta-amino ketones according to the invention of general formula IIb, as shown in reaction diagram IVc 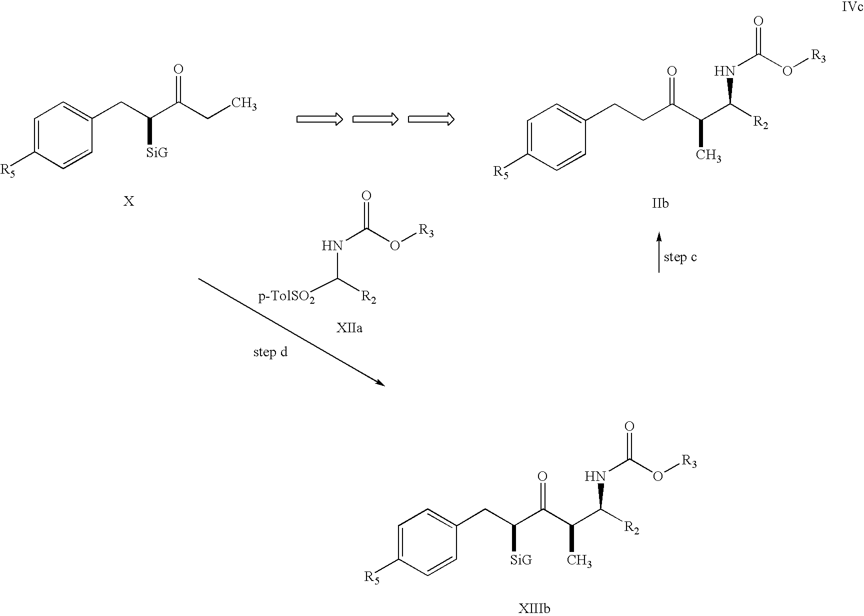
wherein GSi=tert.-hexyl-(CH₂)₂Si,
characterised in that
in step d:
   the temperature is kept at <−70° C.,
   a non-polar organic solvent is used,
   HMPA is used, and
   the reaction takes place in the presence of a base;
in step c:
   the temperature is kept at <−70° C.,
   a non-polar organic solvent is used,
   TBAF or HF are used, and
   the reaction takes place at a pH <7.

It is particularly preferred for reaction diagrams IV, Iva, IVb and IVc if
in step d:
   the temperature is kept at <−78° C., and/or
   THF is used as the non-polar organic solvent, HMPA is used, and/or
   a lithium base, preferably LDA is used as the base;
      and/or
in step c:
   the temperature is kept at <−78° C., and/or
   THF is used as the non-polar organic solvent, and/or
   NH₄F is used to adjust a pH <7, and TBAF or HF is used.

The invention further relates to a process for producing the compounds according to the invention with a free NH₂ group in which, on completion of one of processes III, IIIa, IIIb, IV, IVa, IVb or IVc, in which R₃ corresponds to tert.-butyl, the resulting compounds are reacted with trifluoroacetic acid and the solvent is removed, so the compound according to the invention with a free NH₂ group is produced in the form of the triflate.

The invention further relates to another process for producing the compounds according to the invention with a free NH₂ group in which, on completion of one of processes III, IIIa, IIIb, IV, IVa, IVb or IVc, in which R₃ does not correspond to tert.-butyl, the compounds produced are reacted with HCl and the solvent is removed.

The substances according to the invention are toxicologically safe, so they may be used as a pharmaceutical active ingredient in pharmaceutical compositions. The invention therefore further relates to pharmaceutical compositions containing at least one beta-amino ketone according to the invention and optionally further active ingredients, auxiliaries and/or additives.

Pharmaceutical compositions according to the invention contain, in addition to at least one substituted 4-aminocyclohexanol derivative according to the invention, optionally suitable additives and/or auxiliary agents such as, for example, excipients, fillers, solvents, diluents, dyes and/or binders. The pharmaceutical compositions of the invention can be administered as liquid pharmaceutical preparations in the form of injection solutions, drops or syrups, or as semi-solid pharmaceutical preparations in the form of granules, tablets, pellets, patches, capsules, plasters or aerosols. The choice of auxiliary agents, etc. and the quantities thereof to be used depend on whether the pharmaceutical preparation is to be administered orally, perorally, parenterally, intravenously, intraperitoneally, intradermally, intramuscularly, intranasally, buccally, rectally or topically, for example to the skin, the mucus membranes or the eyes. Preparations in the form of tablets, dragees, capsules, granules, drops and syrups are suitable for oral administration. Solutions, suspensions, easily reconstitutable dry preparations and sprays are suitable for parenteral, topical and inhalative administration. Substituted 4-aminocyclohexanol derivatives according to the invention in a depot, in dissolved form or in a plaster, optionally with the addition of agents to promote skin penetration, are suitable percutaneous application preparations. Orally or percutaneously applicable preparation forms can release the substituted 4-aminocyclohexanol derivatives according to the invention after a delay. In principle, other active ingredients known to the person skilled in the art can be added to the pharmaceutical preparations according to the invention.

The quantity of active ingredient to be administered to the patient varies depending on the weight of the patient, the method of administration, the indication, and the severity of the illness. Conventionally, 0.005 to 1,000 mg/kg, preferably 0.05 to 5 mg/kg of at least one substituted 4-aminocyclohexanol derivative according to the invention are administered.

The invention further relates to the use of a beta-amino ketone according to the invention for the treatment of pain, anxiety, depression or epilepsy, in particular pain.

The invention is further illustrated by the following examples, without being restricted thereto.

### EXAMPLES

The following examples illustrate processes according to the invention and compounds produced therewith. The chemicals and solvents used in the examples were acquired commercially from conventional suppliers (Acros, Avocado, Aldrich, Fluka, Lancaster, Maybridge, Merck, Sigma, TCI, etc.) or synthesised.

### Example 1

The following substances were produced by the processes mentioned above in the main part, in particular by processes IIIa and IVa and IVc—in particular under the preferred conditions:
[5-(4-tert-butyl-phenyl)-1-isopropyl-2-methyl-3-oxo-pentyl]-carbamic acid benzylester,
[1-benzyl-5-(4-bromo-phenyl)-2-methyl-3-oxo-pentyl]-carbamic acid benzylester,
[5-(4-bromo-phenyl)-2-methyl-3-oxo-1-phenyl-pentyl]-carbamic acid tert-butylester,
[5-(4-bromo-phenyl)-1-furan-2-yl-2-methyl-3-oxo-pentyl]-carbamic acid tert-butylester,
[5-(4-bromo-phenyl)-1-(4-tert-butyl-phenyl)-2-methyl-3-oxo-pentyl]-carbamic acid tert-butylester,
[5-(4-bromo-phenyl)-1-(4-methoxy-phenyl)-2-methyl-3-oxo-pentyl]-carbamic acid tert-butylester,
[5-(4-bromo-phenyl)-2-methyl-3-oxo-1-p-tolyl-pentyl]-carbamic acid tert-butylester,
[5-(4-bromo-phenyl)-2-methyl-3-oxo-pentyl]-carbamic acid tert-butylester,
[5-(4-tert-butyl-phenyl)-1-furan-2-yl-2-methyl-3-oxo-pentyl]-carbamic acid tert-butylester,
[5-(4-methoxy-phenyl)-2-methyl-3-oxo-1-phenyl-pentyl]-carbamic acid tert-butylester,
(2-methyl-3-oxo-1-phenyl-heptyl)-carbamic acid tert-butylester
[5-(4-tert-butyl-phenyl)-2-methyl-3-oxo-1-phenyl-pentyl]-carbamic acid tert-butyl ester
[5-(4-methoxyphenyl)-2-methyl-3-oxo-1-(4-trifluoromethyl-phenyl)pentyl]carbamic acid tert-butylester

Procedure

General Directions for Step A (IIIb) (AAV1)

One equivalent of ketone X (diluted with 1 ml abs. THF per mmole) was added dropwise to 1.2 equivalents of LDA (in 5 ml abs. THF and 1.15 ml abs. HMPA per mmole diisopropylamine) under a protective gas atmosphere at −78° C. 1.4 equivalents of undiluted trimethylsilylchloride were then added (8 to 12 minutes). After heating to ambient temperature (approx. 1 hour), excess base was destroyed by addition of saturated aqueous sodium hydrogen carbonate solution. The silylenol ether was extracted with n-pentane, and the organic phase was washed with saturated aqueous sodium chloride solution. Drying over magnesium sulfate, filtration and removal of the solvent on a rotary evaporator yielded the crude silylenol ether XIa.

General Directions for Step B (Diagram IIIb, Method A) (AAV2)

1.2 equivalents of KH were added to a solution of 1.1 equivalents of α-(N-alkoxycarbonyl)amine sulfone XIIa in abs. dichloromethane (5 ml abs. CH₂Cl₂ per mmole), and the mixture was stirred for 1 hour at ambient temperature. After cooling the solution to −79° C., 1.5 equivalents of a 1.0 N titanium tetrachloride dichloromethane solution were added dropwise. After 20 minutes, the mixture was cooled to −90° C. and 1 equivalent of the (S,Z)-silylenol ether XIa diluted with abs. dichloromethane (1 ml abs. CH₂Cl₂ per mmole) was added dropwise over 15 minutes. After stirring for 75 minutes, with the temperature rising to −78° C. again, the reaction was terminated by dropwise addition of an aqueous saturated sodium hydrogen carbonate solution. The solution, which had been heated to ambient temperature, was extracted with dichloromethane. The combined organic phases were washed with saturated aqueous sodium chloride solution. After drying over magnesium sulfate, filtration and removal of the solvent on the rotary evaporator, product XIIIa was purified by column chromatography.

General Direction for Step B (Diagram IIIb, Method B) (AAV3)

1.5 equivalents of a 1.0 N titanium tetrachloride dichloromethane solution or of a 0.5 N tin tetrachloride dichloromethane solution were added dropwise to a solution of 1.1 equivalents of α-(N-alkoxycarbonyl)amine sulfone XIIa in abs. dichloromethane (5 ml abs. CH₂Cl₂ per mmole), and the mixture was stirred for 20 minutes. After cooling to −90° C., 1 equivalent of the (S,Z)-silylenol ether XIa diluted with abs. dichloromethane (1 ml abs. CH₂Cl₂ per mmole) was added dropwise over 15 minutes. After stirring for 75 minutes, with the temperature rising to −78° C. again, the reaction was terminated by dropwise addition of an aqueous saturated sodium hydrogen carbonate solution. The solution, which had been heated to ambient temperature, was extracted with dichloromethane. The combined organic phases were washed with saturated aqueous sodium chloride solution. After drying over magnesium sulfate, filtration and removal of the solvent on the rotary evaporator, product XIIIa was purified by column chromatography.

General Directions for Step D (Diagram IVa, IVc) (AAV4)

One equivalent of ketone V or X (1 ml abs. THF per mmole) was added dropwise to 2.5 equivalents of LDA in abs. Tetrahydrofuran (in 5 ml abs. THF and 1.15 ml abs. HMPA per mmole diisopropylamine) under a protective gas atmosphere at −79° C., and the mixture was metallised for 5 minutes. 1.5 equivalents of α-(N-alkoxycarbonyl)amine sulfone VII or XIIa were dissolved in a little abs. tetrahydrofuran (1 ml abs. THF per mmole) and added dropwise to the reaction solution. After stirring for 25 minutes at −78° C., aqueous saturated sodium hydrogen carbonate solution was added dropwise. The aqueous phase was extracted twice with n-pentane, and the combined organic phases were washed with saturated aqueous sodium chloride solution. After drying over magnesium sulfate and removal of the solvent on a rotary evaporator, the product VIIIb or XIIIb was purified by column chromatography. Further purification and isolation of the excess diastereomer was effected by preparatory HPLC.

General Directions for Step C (Diagram IIIb, IVa, IVc) (AAV5)

One equivalent of β-amino ketone XIIIa, VIIIb or XIIIb was dissolved in abs. tetrahydrofuran (10 ml abs. THF per mmole) and reacted with 50 equivalents of ammonium fluoride and 1.5 to 1.7 equivalents of 1.0 N tetrabutyl ammonium fluoride solution at −78° C. After stirring at −78° C. for 1.0 to 1.5 hours, the mixture was heated to room temperature. The mixture was worked up by addition of pH 6 buffer, extraction in n-pentane or diethyl ether, drying over magnesium sulfate and removal of the solvent on a rotary evaporator. The mixture was purified by crystallisation from n-pentane, diethyl ether or dichloromethane at −20° C. The β-amino ketones IIa, Ib and IIb were obtained as colorless solids.

In particular:

Examples According to AAV 4

### Example 2

### (2R,1R)-[5-(4-bromophenyl)-2-methyl-3-oxo-1-phenyl-pentyl]carbamic acid tert-butylester

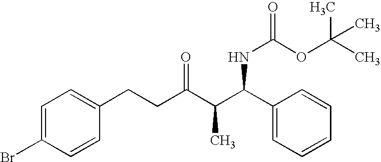

| | | |
|---|---|---|
| Yield: | 194 mg | (87% of theor.) |
| DC: | R_{f} = 0.20 | (n-pentane/Et₂O: 4/1) |
| Melting point | 129° C. | |
| Excess diastereomer: | de > 96% | (NMR, HPLC) |
| Excess enantiomer: | ee > 96% | (HPLC) |
| Amount of rotation: | [α]_{D}²⁵ = −2.9 | (CHCl₃, c = 1.00) |

¹H-NMR spectrum (400 MHz, (CD₃)₂CO):

δ=1.07 (d, 3H, J=6.9, CHCH₃), 1.38 (s, 9H, OC(CH₃)₃), 2.40-2.90 (kB, 4H, CH₂CH₂), 3.17 (qd, 1H, J=8.0/6.9, CHCH₃), 5.04 (m, 1H, CHNH), 6.58 (d, 1H, J=9.3, NH), 7.02 (d, 2H, J=8.3, oCH^{Ph}), 7.20-7.50 (kB, 7H, CH^{Ar}—C₆H₄Br, m/pCH^{Ph}) ppm.

¹³C-NMR spectrum (75 MHz, CDCl₃):

δ=12.3 (CH₃), 28.3 (OC(CH₃)₃), 28.7 (CH₂C₆H₄Br), 43.3 (CH₂CO), 52.0 (CHCH₃), 55.0 (CHNH), 79.8 (OC(CH₃)₃), 119.7 (C^{Ar}—C₆H₄Br), 126.6 (pCH^{Ph}), 127.5 (oCH^{Ph}), 128.3 (mCH^{Ph}), 130.1 (CH^{Ar}—C₆H₄Br), 131.4 (CH^{Ar}—C₆H₄Br), 140.0 (C^{Ph}), 140.4 (C^{Ar}—C₆H₄Br), 155.2 (OC═O), 210.7 (C═O) ppm.

IR spectrum (KBr pressed pellet):

\tilde{ν}=3389 (m), 3359 (s), 3063 (w), 3034 (w), 3007 (w), 2977 (m), 2961 (m), 2931 (m), 2875 (w), 2187 (w), 1897 (w), 1710 (s), 1686 (s), 1586 (w), 1523 (s), 1490 (m), 1453 (m), 1389 (m), 1368 (m), 1292 (m), 1252 (m), 1173 (s), 1120 (m), 1080 (m), 1070 (m), 1041 (w), 1011 (m), 999 (m), 955 (w), 941 (w), 904 (w), 887 (w), 870 (w), 812 (m), 796 (w), 776 (w), 755 (m), 703 (m), 663 (w), 626 (w) cm⁻¹.

Mass spectrum (El, 70 eV):

m/z (r.l. %)=446 (1, M⁺), 391 (7), 389 (7), 211 (4), 206 (27), 171 (10), 169 (7), 151 (9), 150 (100), 147 (4), 134 (4), 132 (5), 118 (14), 117 (7), 107 (7), 106 (81), 104 (9), 79 (5), 77 (5), 57 (5).

Elemental analysis (C₂₃H₂₈NO₃Br, 446.381):

calc.: C=61.89; H=6.32; N=3.14. found: C=62.02; H=6.37; N=3.07.

### Example 3

### (2R,1R)-[5-(4-tert-butylphenyl)-2-methyl-3-oxo-1-phenylpentyl]carbamic acid tert-butylester

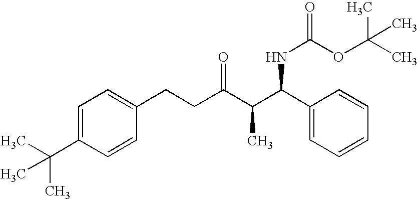

| | | |
|---|---|---|
| Yield: | 169 mg | (85% of theor.) |
| DC: | R_{f} = 0.22 | (n-pentane/Et₂O: 4/1) |
| Melting point: | 70-72° | |
| Excess diastereomer: | de ≧ 96% | (NMR) |
| Excess enantiomer: | ee ≧ 96% | (per de (S,R,R)-36b) |
| Amount of rotation: | [α]_{D}²⁴ = −2.3 | (CHCl₃, c = 1.00) |

¹H-NMR spectrum (300 MHz, (CD₃)₂CO):

δ=1.05 (d, 3H, J=6.9, CHCH₃), 1.27 (s, 9H, C(CH₃)₃), 1.37 (s, 9H, OC(CH₃)₃), 2.40-2.90 (kB, 4H, CH₂CH₂), 3.16 (m, 1H, CHCH₃), 5.05 (m, 1H, CHNH), 6.54 (m, 1H, NH), 6.99 (d, 2H, J=8.2, oCH^{Ph}), 7.10-7.40 (kB, 7H, CH^{Ar}—C₆H₄C(CH₃)₃, m/pCH^{Ph}) ppm.

¹³C-NMR spectrum (75 MHz, (CD₃)₂CO):

δ=11.6 (CH₃), 27.1 (C(CH₃)₃), 27.9 (CH₂C₆H₄C(CH₃)₃), 30.2 (OC(CH₃)₃), 33.3 (C(CH₃)₃), 43.2 (CH₂CO), 51.1 (CHCH₃), 55.7 (CHNH), 77.4 (OC(CH₃)₃), 124.4 (CH^{Ar}—C₆H₄C(CH₃)₃), 126.4 pCH^{Ph}), 126.4 (oCH^{Ph}), 127.2 (mCH^{Ph}), 127.7 (CH^{Ar}—C₆H₄C(CH₃)₃), 137.7 (C^{Ar}—C₆H₄C(CH₃)₃), 141.6 (C^{Ph}), 147.7 (C^{Ar}—C₆H₄C(CH₃)₃), 154.7 (OC═O), 209.8 (C═O) ppm.

IR spectrum (KBr pressed pellet):

\tilde{ν}=3377 (m), 2964 (m), 2932 (s), 2873 (m), 1714 (s), 1683 (s), 1522 (s), 1456 (m), 1391 (m), 1364 (m), 1291 (m), 1260 (m), 1178 (s), 1118 (w), 1078 (w), 1038 (w), 1014 (w), 996 (w), 899 (w), 871 (w), 826 (w), 757 (w), 703 (m), 628 (w), 599 (w), 565 (w), 521 (w) cm⁻¹.

Mass spectrum (El, 70 eV):

m/z (r. l. %)=423 (5.0, M⁺), 368 (6), 367 (22), 306 (5), 218 (6), 206 (15), 189 (6), 161 (5), 160 (10), 151 (9), 150 (100), 147 (18), 145 (8), 134 (5), 133 (13), 132 (8), 118 (9), 117 (8), 107 (7), 106 (84), 91 (5), 57 (43).

Elemental analysis (C₂₇H₃₇NO₃, 423.597):

calc.: C=76.56; H=8.80; N=3.31. found: C=76.19; H=9.01; N=3.06.

### Example 4

### (2R,1R)-[5-(4-methoxyphenyl)-2-methyl-3-oxo-1-phenylpentyl] carbamic acid tert-butylester

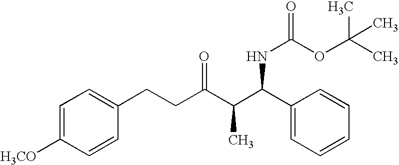

| | | |
|---|---|---|
| Yield: | 279 mg | (88% of theor.) |
| DC: | R_{f} = 0.15 | (n-pentane/Et₂O: 4/1) |
| Melting point: | 127° C. | |
| Excess diastereomer: | de ≧ 96% | (NMR) |
| Excess enantiomer: | ee ≧ 96% | (per de (S,R,R)-36c) |
| Amount of rotation: | [α]_{D}²⁴ = −5.8 | (CHCl₃, c = 1.00) |

¹H-NMR spectrum (300 MHz, (CD₃)₂CO):

δ=1.07 (d, 3H, J=6.9, CHCH₃), 1.38 (s, 9H, OC(CH₃)₃), 2.40-2.90 (kB, 4H, CH₂CH₂), 3.16 (m, 1H, CHCH₃), 3.74 (s, 3H, OCH₃), 5.05 (M, 1H, CHNH), 6.54 (d, 1H, J=9.9, NH), 6.50-7.40 (kB, 9H, CH^{Ar}—C₆H₄OCH₃, CH^{Ph}) ppm.

¹³C-NMR spectrum (75 MHz, (CD₃)₂CO):

δ=13.1 (CH₃), 28.6 (OC(CH₃)₃), 39.1 (CH₂C₆H₄OCH₃), 44.9 (CH₂CO), 52.6 (CHCH₃), 55.4 (CHNH), 57.2 (OCH₃), 78.9 (OC(CH₃)₃), 114.5 (CH^{Ar}—C₆H₄OCH₃), 127.8 (pCH^{Ph}), 127.9 (oCH^{Ph}), 129.1 (mCH^{Ph}), 129.9 (CH^{Ar}—C₆H₄OCH₃), 134.1 (C^{Ph}), 143.0 (C^{Ar}—C₆H₄OCH₃), 156.2 (OC═O), 158.8 (C^{Ar}—C₆H₄OCH₃), 211.3 (C═O) ppm.

IR spectrum (KBr pressed pellet):

\tilde{ν}=3376 (s), 3002 (m), 2981 (m), 2964 (m), 2931 (m), 2872 (w), 2834 (w), 2344 (w), 1707 (s), 1679 (s), 1610 (m), 1582 (w), 1513 (s), 1444 (m), 1410 (m), 1391 (m), 1364 (m), 1363 (m), 1332 (w), 1301 (m), 1291 (m), 1269 (m), 1241 (s), 1179 (s), 1110 (m), 1081 (m), 1039 (m), 1013 (m), 997 (m), 900 (w), 875 (w), 827 (m), 816 (m), 780 (w), 762 (m), 753 (w), 702 (s), 631 (w), 608 (w) cm⁻¹.

Mass spectrum (El, 70 eV):

m/z (r. l. %)=397 (6, M⁺), 342 (9), 341 (38), 280 (5), 206 (8), 192 (13), 191 (43), 173 (5), 163 (22), 162 (7), 161 (7), 151 (8), 150 (80), 135 (21), 134 (30), 132 (8), 122 (8), 121 (87), 118 (7), 117 (7), 107 (9), 106 (100), 105 (10), 91 (10), 79 (10), 78 (5), 77 (10), 57 (52).

Elemental analysis (C₂₄H₃₁NO₄, 397.515):

| | | | |
|---|---|---|---|
| calc.: | C = 72.52 | H = 7.86 | N = 3.52 |
| found: | C = 72.43 | H = 7.66 | N = 3.39 |

### Example 5

### (2R,1R)-[5-(4-tert-butylphenyl)-1-furan-2-yl-2-methyl-3-oxopentyl)carbamic acid tert-butylester

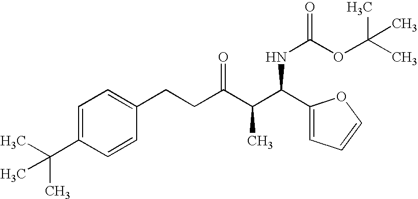

| | | |
|---|---|---|
| Yield: | 144 mg | (87% of theor.) |
| DC: | R_{f} = 0.31 | (n-pentane/Et₂O: 4/1) |
| Melting point: | 85° C. | |
| Excess diastereomer: | de ≧ 96% | (NMR) |
| Excess enantiomer: | ee ≧ 96% | (per de (S,R,R)-36e) |
| Amount of rotation: | [α]_{D}²⁴ = +24.0 | (CHCl₃, c = 0.50) |

¹H-NMR spectrum (400 MHz, (CD₃)₂CO):

δ=1.06 (d, 3H, J=7.1, CHCH₃), 1.29 (s, 9H, C(CH₃)₃), 1.40 (s, 9H, OC(CH₃)₃), 2.65-2.93 (kB, 4H, CH₂CH₂), 3.19 (qd, 1H, J=8.2/6.9, CHCH₃), 5.16 (m, 1H, CHNH), 6.20 (m, 1H, CH^{Ar}—C₄H₃O), 6.33 (M, 1H, CH^{Ar}—C₄H₃O), 4.40 (m, 1H, NH), 7.00 (d, 2H, J=8.2, CH^{Ar}—C₆H₄C(CH₃)₃), 7.29 (d, 2H, J=8.2, CH^{Ar}—C₆H₄C(CH₃)₃), 7.43 (m, 1H, CH^{Ar}—C₄H₃O) ppm.

¹³C-NMR spectrum (100 MHz, (CD₃)₂CO):

δ=12.9 (CH₃), 28.5 (OC(CH₃)₃), 29.6 (CH₂C₆H₄C(CH₃)₃), 31.7 (C(CH₃)₃), 34.8 (C(CH₃)₃), 44.0 (CH₂CO), 50.3 (CHCH₃), 51.2 (CHNH), 79.2 (OC(CH₃)₃), 107.0 (CH^{Ar}—C₄H₃O), 111.1 (CH^{Ar}—C₄H₃O), 125.9 (CH^{Ar}—C₆H₄C(CH₃)₃), 128.8 (CH^{Ar}—C₆H₄C(CH₃)₃), 139.3 (C^{Ar}—C₆H₄C(CH₃)₃), 142.6 (CH^{Ar}—C₄H₃O), 149.2 (C^{Ar}—C₆H₄C(CH₃)₃), 155.4 (C^{Ar}—C₄H₃O), 156.2 (OC═O), 210.6 (C═O) ppm.

IR spectrum (KBr pressed pellet):

\tilde{ν}=3324 (s), 3123 (w), 2961 (s), 2871 (m), 2369 (w), 2344 (w), 2287 (w), 1904 (w), 1706 (s), 1684 (s), 1522 (s), 1454 (m), 1408 (m), 1391 (m), 1367 (s), 1322 (m), 1279 (s), 1243 (m), 1174 (s), 1077 (m), 1039 (m), 1013 (m), 1001 (m), 942 (w), 913 (w), 884 (w), 865 (w), 824 (m), 789 (w), 732 (m), 702 (w), 660 (m), 599 (w), 564 (m), 520 (w) cm⁻¹.

Mass spectrum (El, 70 eV):

m/z (r. l. %)=413 (3, M⁺), 357 (19), 313 (6), 312 (7), 297 (5), 296 (6), 196 (13), 167 (5), 166 (9), 149 (8), 147 (28), 145 (8), 141 (10), 133 (17), 132 (8), 131 (12), 125 (6), 124 (17), 123 (6), 122 (5), 119 (5), 117 (10), 111 (5), 109 (7), 108 (29), 107 (5), 105 (7), 97 (13), 96 (100), 95 (7), 91 (6), 85 (8), 83 (7), 81 (6), 74 (8), 71 (11), 69 (11), 57 (97), 56 (7), 55 (16), 45 (6).

Elemental analysis (C₂₅H₃₅NO₄, 413.558):

| | | | |
|---|---|---|---|
| calc.: | C = 72.61 | H = 8.53 | N = 3.38 |
| found: | C = 72.73 | H = 8.91 | N = 3.25 |

### Example 6

### (2R,1R)-[5-(4-tert-butylphenyl)-1-isopropyl-2-methyl-3-oxo-pentyl]-carbamic acid benzylester

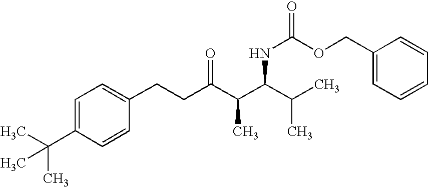

| | | |
|---|---|---|
| Yield: | 194 mg | (88% of theor.) |
| DC: | R_{f} = 0.20 | (n-pentane/Et₂O: 4/1) |
| Melting point: | 47° C. | |
| Excess diastereomer: | de ≧ 85% | (NMR) |
| Excess enantiomer: | not determined | |
| Amount of rotation: | [α]_{D}²⁴ = −13.5 | (CHCl₃, c = 1.05) |

¹H-NMR spectrum (400 MHz, (CD₃)₂CO):

δ=0.89 (d, 3H, J=6.9, CH₃CHCH₃), 0.92 (d, 3H, J=6.9, CH₃CHCH₃), 0.99 (d, 3H, J=6.9, CHCH₃), 1.29 (s, 9H, C(CH₃)₃), 1.65 (m, 1H, CH₃CHCH₃), 2.76-3.02 (kB, 5H, CH₂CH₂, CHCH₃), 3.92 (m, 1H, CHNH), 5.03 (d, 1H, J=12.6, CHHPh), 5.05 (d, 1H, J=12.6, CHHPh), 6.13 (d, 1H, J=10.2, NH), 7.14 (d, 2H, J=8.2, CH^{Ar}—C₆H₄C(CH₃)₃), 7.26-7.36 (kB, 7H, CH^{Ar}—C₆H₄C(CH₃)₃, CH^{Ph}) ppm.

¹³C-NMR spectrum (100 MHz, (CD₃)₂CO):

δ=11.3 (CH₃), 18.4 (CH₃CHCH₃), 20.5 (CH₃CHCH₃), 29.8 (CH₂C₆H₄), 31.2 (C(CH₃)₃), 31.6 (C(CH₃)₃), 34.8 (CH₃CHCH₃), 43.4 (CH₂CO), 48.9 (CHCH₃), 58.6 (CHNH), 66.3 (CH₂Ph), 125.7 (CH^{Ar}—C₆H₄C(CH₃)₃), 128.2 (CH^{Ph}), 128.3 (CH^{Ph}) 128.7 (CH^{Ph}), 128.9 (CH^{Ar}—C₆H₄C(CH₃)₃), 138.3 (C^{Ar}—C₆H₄C(CH₃)₃), 139.3 (C^{Ph}), 148.9 (C^{Ar}—C₆H₄C(CH₃)₃), 157.3 (OC═O), 211.2 (C═O) ppm.

IR spectrum (KBr pressed pellet):

\tilde{ν}=3345 (m), 3032 (w), 2962 (s), 2875 (m), 2208 (w), 1704 (s), 1687 (s), 1533 (s), 1372 (m), 1309 (s), 1245 (s), 1177 (w), 1155 (w), 1121 (m), 1092 (m), 1045 (w), 1020 (m), 993 (m), 823 (m), 779 (w), 741 (m), 724 (m), 696 (m), 563 (w), 463 (w) cm⁻¹.

Mass spectrum (El, 70 eV):

m/z (r.l. %)=423 (25, M⁺), 380 (5), 288 (10), 241 (6), 236 (11), 217 (5), 206 (13), 192 (6), 189 (10), 172 (12), 163 (5), 162 (25), 161 (8), 154 (5), 148 (8), 147 (17), 117 (6), 116 (24), 92 (8), 91 (100), 72 (27), 57 (14).

Elemental analysis (C₂₇H₃₇NO₃, 423.597):

| | | | |
|---|---|---|---|
| calc.: | C = 76.56 | H = 8.80 | N = 3.31 |
| found: | C = 76.50 | H = 8.97 | N = 3.19 |

### Example 8

### (2R,1R)-[5-(4-methoxyphenyl)-2-methyl-3-oxo-1-(4-trifluoromethyl-phenyl)pentyl]carbamic acid tert-butylester

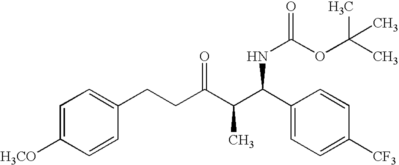

| | | |
|---|---|---|
| Yield: | 112 mg | (80% of theor.) |
| DC: | R_{f} = 0.13 | (n-pentane/Et₂O: 4/1) |
| Melting point: | 151° C. | |
| Excess diastereomer: | de ≧ 96% | (NMR) |
| Excess enantiomer: | ee ≧ 96% | (per de (S,R,R)-36i) |
| Amount of rotation: | [α]_{D}²⁴ = +9.1 | (CHCl₃, c = 1.00) |

¹H-NMR spectrum (300 MHz, (CD₃)₂CO):

δ=1.09 (d, 3H, J=6.6, CHCH₃), 1.38 (s, 9H, OC(CH₃)₃), 2.40-2.90 (kB, 4H, CH₂CH₂), 3.23 (qd, 1H, J=8.0/6.8, CHCH₃), 3.75 (s, 3H, OCH₃), 5.15 (m, 1H, CHNH), 6.70 (d, 1H, J=9.3, NH), 6.78 (d, 2H, J=8.2, CH^{Ar}—C₆H₄OCH₃), 6.99 (d, 2H, J=8.2, CH^{Ar}—C₆H₄CF₃), 7.60 (d, 2H, J=8.2, CH^{Ar}—C₆H₄OCH₃), 7.67 (d, 2H, J=8.2, CH^{Ar}—C₆H₄CF₃) ppm.

¹³C-NMR spectrum (75 MHz, CDCl₃):

δ=13.1 (CH₃), 28.5 (OC(CH₃)₃), 29.1 (CH₂C₆H₄), 44.7 (CH₂CO), 52.1 (CHCH₃), 55.3 (CHNH), 56.8 (OCH₃), 79.2 (OC(CH₃)₃), 114.5 (CH^{Ar}—C₆H₄OCH₃), 126.0 (CH^{Ar}—C₆H₄CF₃), 128.7 (CH^{Ar}—C₆H₄CF₃), 129.3 (CF₃) 129.7 (C^{Ar}—H₄CF₃), 129.9 (CH^{Ar}—C₆H₄OCH₃), 134.0 (C^{Ar}—C₆H₄CF₃), 147.8 (C^{Ar}—C₆H₄OCH₃), 156.2 (OC═O), 158.9 (C^{Ar}—C₆H₄OCH₃), 212.5 (C═O) ppm.

IR spectrum (KBr pressed pellet):

\tilde{ν}=3367 (s), 3059 (w), 2982 (m), 2933 (m), 2880 (w), 2834 (m), 2068 (w), 1886 (w), 1707 (s), 1681 (s), 1613 (m), 1584 (m), 1531 (s), 1514 (s), 1461 (m), 1445 (m), 1423 (m), 1393 (m), 1330 (m), 1300 (m), 1249 (m), 1179 (m), 1162 (m), 1128 (m), 1072 (m), 1038 (m), 1018 (m), 997 (m), 910 (w), 875 (m), 848 (m), 826 (m), 769 (w), 752 (w), 734 (w), 714 (w), 667 (w), 644 (m), 633 (m), 609 (m), 543 (m) cm⁻¹.

Mass spectrum (El, 70 eV):

m/z (r.l. %)=465 (4, M⁺), 410 (6), 409 (29), 218 (14), 192 (10), 191 (35), 175 (4), 174 (45), 173 (6), 164 (4), 163 (33), 161 (4), 135 (18), 134 (14), 122 (9), 121 (100), 105 (4), 91 (5), 77 (5), 57 (52).

Elemental analysis (C₂₅H₃₀NO₄F₃, 465.512):

| | | | |
|---|---|---|---|
| calc.: | C = 64.50 | H = 6.50 | N = 3.01 |
| found: | C = 64.22 | H = 6.54 | N = 3.07 |

### Example 9

### (2R,1R)-(2-methyl-3-oxo-1-phenyl-heptyl)carbamic acid tert-butyl ester

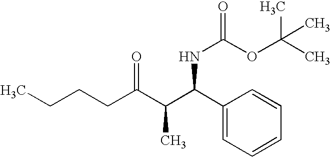

| | | |
|---|---|---|
| Yield: | 118 mg | (92% of theor.) |
| DC: | R_{f} = 0.24 | (n-pentane/Et₂O: 4/1) |
| Melting point: | 101° C. | |
| Excess diastereomer: | de ≧ 96% | (NMR) |
| Excess enantiomer: | ee ≧ 96% | (per de (S,R,R)-36j) |
| Amount of rotation: | [α]_{D}²⁴ = −18.3 | (CHCl₃, c = 0.91) |

¹H-NMR spectrum (300 MHz, (CD₃)₂CO):

δ=0.76 (t, 3H, J=7.1, CH₂CH₃), 1.10 (kB, 5H, CH₂CH₃), 1.28 (m, 2H, CH₂CH₂CH₃), 1.36 (s, 9H, OC(CH₃)₃), 2.08-2.45 (kB, 2H, CH₂CO), 3.15 (m, 1H, CHCH₃), 4.99 (m, 1H, CHNH), 6.50 (m, 1H, NH), 7.15-7.45 (kB, 5H, CH^{Ph}) ppm.

¹³C-NMR spectrum (75 MHz, CDCl₃):

δ=12.6 (CH₂CH₃), 14.2 (CHCH₃), 22.5 (CH₂CH₃), 25.7 (CH₂CH₂CH₃), 28.6 (OC(CH₃)₃), 41.9 (CH₂CO), 52.0 (CHCH₃), 56.3 (CHNH), 80.1 (OC(CH₃)₃), 127.0 (CH^{Ph}), 127.7 (CH^{Ph}), 128.9 (CH^{Ph}), 141.0 (C^{Ph}), 155.5 (OC═O), 212.6 (C═O) ppm.

IR spectrum (KBr pressed pellet):

\tilde{ν}=3380 (s), 3067 (w), 3009 (m), 2977 (m), 2963 (m), 2933 (m), 2873 (m), 2166 (w), 1709 (s), 1682 (s), 1522 (s), 1457 (m), 1408 (m), 1366 (m), 1291 (m), 1263 (m), 1177 (s), 1125 (m), 1077 (m), 1035 (m), 1011 (m), 968 (w), 927 (w), 901 (m), 831 (w), 780 (w), 757 (m), 703 (m), 629 (w), 596 (w), 518 (w) cm⁻¹.

Mass spectrum (Cl, isobutane):

m/z (r.l. %)=320 (90, M⁺+1), 302 (3), 265 (17), 262 (100), 258 (3), 206 (11).

Elemental analysis (C₁₉H₂₉NO₃, 319.445):

| | | | |
|---|---|---|---|
| calc.: | C = 71.44 | H = 9.15 | N = 4.38 |
| found: | C = 71.34 | H = 9.21 | N = 4.22 |

### Example 10

### (2R)-[5-(4-bromophenyl)-2-methyl-3-oxo-pentyl]carbamic acid tert-butyl ester

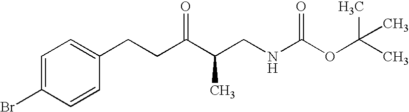

| | | |
|---|---|---|
| Yield: | 158 mg | (84% of theor.) |
| DC: | R_{f} = 0.25 | (n-pentane/Et₂O: 4/1) |
| Melting point: | 48° C. | |
| Excess diastereomer: | de ≧ 96% | (NMR, HPLC) |
| Excess enantiomer: | ee ≧ 95% | (HPLC) |
| Amount of rotation: | [α]_{D}²⁵ = −11.0 | (CHCl₃, c = 1.00) |

¹H-NMR spectrum (400 MHz, (CD₃)₂CO):

δ=1.04 (d, 3H, J=7.1, COCHCH₃), 1.40 (s, 9H, OC(CH₃)₃), 2.78-3.35 (kB, 7H, CH₂CH₂C₆H₄Br, CHCH₂NH), 5.98 (s, 1H, NH), 7.21 (d, 2H, J=8.5, CH^{Ar}—C₆H₄Br), 7.44 (d, 2H, J=8.5, CH^{Ar}—C₆H₄Br) ppm.

¹³C-NMR spectrum (100 MHz, (CD₃)₂CO):

δ=12.8 (CH₃), 27.1 (OC(CH₃)₃), 28.0 (CH₂C₆H₄Br), 41.6 (CH₂CO), 42.1 (CH₂NH), 45.7 (CHCH₃), 77.2 (C(CH₃), 118.5 (C^{Ar}—C₆H₄Br), 129.9 (CH^{Ar}—C₆H₄Br), 130.6 (CH^{Ar}—C₆H₄Br), 140.5 (C^{Ar}—C₆H₄Br), 155.2 (OC═O), 210.4 (C═O) ppm.

IR spectrum (KBr pressed pellet):

\tilde{ν}=3373 (s), 2975 (m), 2931 (m), 2873 (m), 1902 (w), 1706 (s), 1684 (s), 1520 (s), 1489 (m), 1459 (m), 1403 (m), 1386 (m), 1367 (m), 1339 (m), 1280 (s), 1250 (s), 1170 (s), 1106 (m), 1071 (m), 1012 (m), 1000 (m), 981 (m}, 937 (m), 889 (w), 864 (w), 815 (m), 782 (m), 604 (m), 515 (m), 462 cm⁻¹.

Mass spectrum (El, 70 eV):

m/z (r.l. %)=370 (2, M⁺), 315 (42), 314 (12), 313 (76), 298 (15), 296 (14), 271 (17), 270 (4), 269 (19), 254 (10), 252 (15), 242 (12), 240 (15), 230 (7), 228 (6), 213 (20), 211 (15), 185 (12), 184 (5), 183 (11), 173 (14), 171 (35), 170 (4), 169 (36), 130 (9), 112 (8), 104 (13), 103 (16), 102 (7), 90 (7), 86 (5), 77 (5), 74 (4), 59 (17), 58 (14), 57 (100), 56 (5).

Elemental analysis (C₁₇H₂₄NO₃Br, 370.283):

| | | | |
|---|---|---|---|
| calc.: | C = 55.14 | H = 6.53 | N = 3.78 |
| found: | C = 55.20 | H = 6.45 | N = 3.65 |

Examples According to AAV1 and AAV2 or AAV3

### Example 11

### (2R,1S)-[5-(4-bromophenyl)-2-methyl-3-oxo-1-phenyl-pentyl]carbamic acid tert-butylester

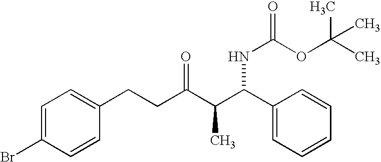

| | | |
|---|---|---|
| Yield: | 106 mg | (95% of theor.) |
| DC: | R_{f} = 0.21 | (n-pentane/Et₂O: 4/1) |
| Melting point: | 148° C. | |
| Excess diastereomer: | de ≧ 98% | (HPLC) |
| Excess enantiomer: | ee > 98% | (HPLC) |
| Amount of rotation: | [α]_{D}²⁴ = −29.0 | (CHCl₃, c = 0.50) |

¹H-NMR spectrum (400 MHz, CDCl₃):

δ=1.14 (d, 3H, J=6.9, CHCH₃), 1.41 (s, 9H, OC(CH₃)₃), 2.15-2.70 (kB, 4H, CH₂CH₂), 3.05 (m, 1H, CHCH₃), 4.81 (m, 1H, CHNH), 5.87 (m, 1H, NH), 6.87 (d, 2H, J=8.3, oCH^{Ph}), 7.10-7.35 (kB, 7H, CH^{Ar}—C₆H₄Br, m/pCH^{Ph}) ppm.

¹³C-NMR spectrum (100 MHz, CDCl₃):

δ=15.1 (CH₃), 28.3 (OC(CH₃)₃), 28.4 (CH₂C₆H₄Br), 44.2 (CH₂CO), 51.0 (COCHCH₃), 57.0 (CHNH), 79.4 (OC(CH₃)₃), 119.6 (C^{Ar}—C₆H₄Br), 125.9 (pCH^{Ph}), 127.1 (oCH^{Ph}), 128.4 (m CH^{Ph}), 129.8 (CH^{Ar}—C₆H₄Br), 131.2 (CH^{Ar}—C₆H₄Br), 139.6 (C^{Ph}), 141.0 (C^{Ar}—C₆H₄Br), 155.3 (OC═O), 213.2 (C═O) ppm.

IR spectrum (KBr pressed pellet):

\tilde{ν}=3371 (s), 3060 (w), 3031 (w), 2979 (m), 2966 (m), 2931 (m), 2875 (w), 2345 (w), 1975 (w), 1892 (w), 1704 (s), 1685 (s), 1588 (w), 1516 (s), 1489 (m), 1458 (m), 1401 (w), 1389 (w), 1367 (m), 1336 (w), 1293 (m), 1251 (m), 1227 (w), 1208 (w), 1171 (m), 1122 (w), 1104 (w), 1083 (w), 1073 (w), 1048 (w), 1012 (m), 997 (w), 928 (w), 908 (w), 879 (w), 866 (w), 839 (w), 826 (w), 794 (w), 757 (m), 738 (w), 705 (m), 631 (w), 581 (w), 558 (w), 519 (w) cm⁻¹.

Mass spectrum (El, 70 eV):

m/z (r.l. %) 446 (1, M⁺), 390 (23), 389 (21), 211 (4), 207 (4), 206 (34), 184 (4), 182 (4), 170 (13), 169 (13), 151 (9), 150 (100), 147 (4), 134 (4), 132 (7), 118 (15), 117 (9), 107 (6), 106 (70), 104 (9), 77 (5), 57 (59).

Elemental analysis (C₂₃H₂₈NO₃Br, 446.381):

| | | | |
|---|---|---|---|
| calc.: | C = 61.89 | H = 6.32 | N = 3.14 |
| found: | C = 61.82 | H = 6.30 | N = 3.00 |

### Example 12

### (2R,1S)-[5-(4-bromophenyl)-1-(4-methoxyphenyl)-2-methyl-3-oxopentyl]-carbamic acid tert-butylester

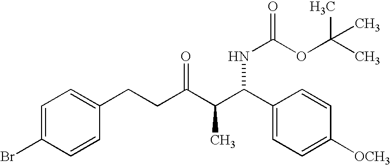

| | | |
|---|---|---|
| Yield: | 177 mg | (93% of theor.) |
| DC : | R_{f} = 0.22 | (n-pentane/Et₂O: 4/1) |
| Melting point: | 176° C. | |
| Excess diastereomer: | de ≧ 96% | (NMR) |
| Excess enantiomer: | ee ≧ 96% | (per de (S,R,S)-37b) |
| Amount of rotation: | [α]_{D}²⁴ = −31.1 | (CHCl₃, c = 1.00) |

¹H-NMR spectrum (400 MHz, CDCl₃):

δ=1.11 (d, 3H, J=6.9, CHCH₃), 1.40 (s, 9H, OC(CH₃)₃), 2.20-2.70 kB, 4H, H₂CH₂), 3.00 (m, 1H, CHCH₃), 3.78 (s, 3H, OCH₃), 4.75 (m, 1H, CHNH), 5.79 (m, 1H, NH), 6.81 (d, 2H, J=8.2, CH^{Ar}—-C₆H₄OCH₃), 6.90 (d, 2H, J=8.2, CH^{Ar—C}₆H₄OCH₃), 7.08 (d, 2H, J=8.5, CH^{Ar}—C₆H₄Br), 7.33 (d, 2H, J=8.5, CH^{Ar}—C₆H₄Br) ppm.

¹³C-NMR spectrum (100 MHz, CDCl₃):

δ=15.0 (CH₃), 28.3 (OC(CH₃)₃), 28.4 (CH₂C₆H₄Br), 44.0 (CH₂CO), 51.1 (COCHCH₃), 55.2 (OCH₃), 56.4 (CHNH), 79.4 (OC(CH₃)₃), 113.8 (CH^{Ar}—-C₆H₄OCH₃), 119.6 (C^{Ar}—C₆H₄Br), 127.0 (CH^{Ar}—C₆H₄OCH₃), 129.9 (CH^{Ar}—C₆H₄Br), 131.2 (CH^{Ar}—C₆H₄Br), 133.1 (C^{Ar}—C614OCH₃), 139.7 (C^{Ar}—C₆H₄Br), 155.5 (OC═O), 158.5 (C^{Ar}—C₆H₄OCH₃), 213.3 (C═O) ppm.

IR spectrum (KBr pressed pellet):

\tilde{ν}=3353 (s), 3034 (w), 2961 (m), 2932 (m), 2872 (m), 2837 (w), 2366 (w), 2285 (w), 1894 (w), 1707 (s), 1683 (s), 1614 (m), 1582 (w), 1527 (s), 1490 (m), 1451 (m), 1369 (m), 1332 (w), 1300 (m), 1253 (s), 1210 (w), 1168 (s), 1121 (w), 1071 (m), 1031 (m), 1010 (m), 915 (w), 888 (w), 871 (w), 847 (w), 824 (m), 813 (m), 778 (w), 736 (w), 711 (w), 659 (w), 636 (w), 586 (w), 546 (w), 516 (w) cm⁻¹.

Mass spectrum (El, 70 eV):

m/z (r.l. %)=476 (1, M⁺), 421 (5), 419 (6), 375 (6), 347 (5), 326 (15), 325 (77), 292 (5), 279 (14), 278 (60), 251 (5), 250 (27), 249 (22), 237 (6), 236 (24), 235 (8), 221 (5), 220 (6), 219 (27), 181 (10), 180 (100), 175 (6), 170 (7), 169 (6), 162 (5), 161 (7), 158 (8), 148 (6), 147 (28), 145 (7), 136 (24), 135 (8), 134 (32), 133 (9), 131 (6), 129 (8), 128 (7), 121 (17), 119 (7), 118 (5), 117 (34), 115 (21), 104 (8), 103 (12), 91 (24), 89 (5), 78 (5), 77 (11), 65 (5), 57 (32), 55 (7).

Elemental analysis (C₂₄H₃₀NO₄Br, 476.407):

| | | | |
|---|---|---|---|
| calc.: | C = 60.51 | H = 6.35 | N = 2.94 |
| found: | C = 60.27 | H = 6.32 | N = 2.82 |

### Example 13

### (2R,1S)-[5-(4-bromophenyl)-1-(4-tert-butylphenyl)-2-methyl-3-oxopentyl]-carbamic acid tert-butylester

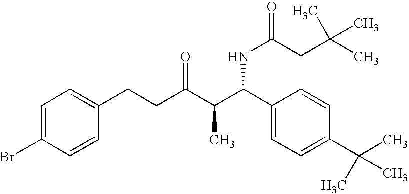

| | | |
|---|---|---|
| Yield: | 136 mg | (90 % of theor.) |
| DC: | R_{f} = 0.22 | (n-pentane/Et₂O: 4/1) |
| Melting point: | 91° C. | |
| Excess diastereomer: | de ≧ 96% | (NMR) |
| Excess enantiomer: | ee ≧ 96% | (per de (S, R, S)-37c) |

| | |
|---|---|
| Amount of rotation: | [α]_{D}²⁴ = −28.6 (CHCl₃, c = 0.50) |

¹H-NMR spectrum (300 MHz, (CD₃)₂CO):

δ=1.05 (d, 3H, J=7.1, CHCH₃), 1.31 (s, 9H, C(CH₃)₃), 1.37 (s, 9H, OC(CH₃)₃), 2.55-3.00 (kB, 4H, CH₂CH₂), 3.13 (m, 1H, CHCH₃), 4.82 (m, 1H, CHNH), 6.51 (m, 1H, NH), 7.10-7.50 (kB, 8H, CH^{Ar}—C₆H₄Br, CH^{Ar}—C₆H₄C(CH₃)₃) ppm.

¹³C-NMR spectrum (75 MHz, (CD₃)₂CO):

δ=15.2 (CH₃), 28.6 (C(CH₃)₃), 29.2 (CH₂C₆H₄Br), 31.6 (OC(CH₃)₃), 34.9 (C(CH₃)₃), 43.8 (CH₂CO), 52.0 (COCHCH₃), 57.7 (CHNH), 78.9 (OC(CH₃)₃), 119.6 (C^{Ar}—C₆H₄Br), 126.0 (CH^{Ar}—C₆H₄C(CH₃)₃), 127.5 (CH^{Ar}—C₆H₄C(CH₃)₃), 131.3 (CH^{Ar}—₆H₄Br), 132.1 (CH^{Ar}—C₆H₄Br), 139.7 (C^{Ar}—C₆H₄C(CH₃)₃), 141.9 (C^{Ar}—C₆H₄Br), 150.7 (C^{Ar}—C₆H₄C(CH₃)₃), 155.7 (OC═O), 212.7 (C═O) ppm.

IR spectrum (KBr pressed pellet):

\tilde{ν}=3390 (m), 2966 (s), 2932 (m), 2871 (m), 2372 (w), 2345 (w), 2287 (w), 1899 (w), 1708 (s), 1689 (s), 1512 (s), 1489 (m), 1457 (m), 1391 (m), 1366 (m), 1310 (w), 1291 (m), 1250 (m), 1231 (w), 1171 (s), 1105 (w), 1073 (m), 1050 (w), 1010 (m), 959 (w), 917 (w), 881 (w), 832 (m), 814 (m), 776 (w), 748 (w), 701 (w), 664 (w), 598 (w), 555 (w), 519 (w), 489 (w) cm⁻¹.

Mass spectrum (El, 70 eV):

m/z (r.l. %)=503 (1, M⁺), 447 (5), 445 (5), 402 (4), 400 (4), 263 (5), 262 (25), 207 (15), 206 (100), 174 (4), 171 (6), 169 (6), 163 (6), 162 (37), 159 (4), 147 (5), 146 (4), 97 (4), 85 (4), 71 (6), 69 (4), 57 (48), 55 (7).

Elemental analysis (C₂₇H₃₆NO₃Br, 502.489):

| | | | |
|---|---|---|---|
| calc.: | C = 64.54 | H = 7.22 | N = 2.79 |
| found: | C = 64.20 | H = 6.84 | N = 2.61 |

### Example 14

### (2R,1S)-[5-(4-bromophenyl)-2-methyl-3-oxo-1-p-tolylpentyl]carbamic acid tert-butylester

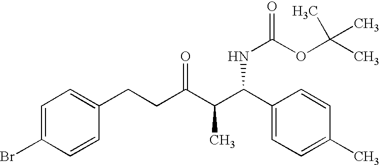

| | | |
|---|---|---|
| Yield: | 127 mg | (92% of theor.) |
| DC: | R_{f} = 0.16 | (n-pentane/Et₂O: 4/1) |
| Melting point: | 159° C. | |
| Excess diastereomer: | de ≧ 96% | (NMR) |
| Excess enantiomer: | ee ≧ 96% | (per de (S, R, S)-37d) |
| Amount of rotation: | [α]_{D}²⁴ = −32.4 | (CHCl₃, c = 0.50) |

¹H-NMR spectrum (400 MHz, CDCl₃):

δ=1.12 (d, 3H, J=6.9, CHCH₃), 1.40 (s, 9H, OC(CH₃)₃), 2.31 (s, 3H, C₆H₄CH₃), 2.15-2.70 (kB, 4H, CH₂CH₂), 3.02 (m, 1H, CHCH₃), 4.77 (m, 1H, CHNH), 5.83 (m, 1H, NH), 6.88 (d, 2H, J=8.2, CH^{Ar}—C₆H₄CH₃), 7.02 (kB, 4H, CH^{Ar}—C₆H₄CH₃, CH^{Ar}—C₆H₄Br), 7.33 (d, 2H, J=8.2, CH^{Ar}—C₆H₄Br) ppm.

¹³C-NMR spectrum (100 MHz, CDCl₃):

δ=15.0 (CHCH₃), 21.0 (C₆H₄CH₃), 28.3 (OC(CH₃)₃), 28.4 (CH₂C₆H₄Br), 44.2 (CH₂CO), 51.1 (COCHCH₃), 56.7 (CHNH), 79.5 (OC(CH₃)₃), 119.7 (C^{Ar}—C₆H₄Br), 125.9 (CH^{Ar}—C₆H₄CH₃), 129.3 (CH^{Ar}—C₆H₄CH₃), 130.0 (CH^{Ar}—C₆H₄Br), 131.4 (CH^{Ar}—C₆H₄Br), 136.9 (C^{Ar}—C₆H₄CH₃), 138.2 (C^{Ar}—C₆H₄CH₃), 139.9 (C^{Ar}—C₆H₄Br), 155.5 (OC═O), 213.6 (C═O) ppm.

IR spectrum (KBr pressed pellet):

\tilde{ν}=3358 (s), 2977 (m), 2929 (m), 2872 (w), 2369 (w), 2285 (w), 1898 (w), 1708 (s), 1683 (s), 1527 (s), 1490 (m), 1455 (m), 1395 (m), 1369 (m), 1332 (m), 1312 (m), 1288 (m), 1254 (m), 1169 (s), 1121 (m), 1071 (m), 1050 (w), 1007 (m), 915 (w), 888 (w), 871 (w), 848 (w), 812 (m), 771 (w), 719 (w), 653 (w), 544 (w), 515 (w) cm⁻¹.

Mass spectrum (El, 70 eV):

m/z (r.l. %)=460 (1, M⁺), 405 (5), 403 (6), 221 (4), 220 (26), 171 (7), 169 (8), 165 (10), 164 (100), 161 (4), 146 (6), 132 (9), 121 (5), 120 (53), 118 (4), 104 (5), 93 (4), 91 (5), 57 (43).

Elemental analysis (C₂₄H₃₀NO₃Br, 460.408):

| | | | |
|---|---|---|---|
| calc.: | C = 62.61 | H = 6.57 | N = 3.04 |
| found: | C = 62.64 | H = 6.45 | N = 2.87 |

### Example 15

### (2R,1S)-[5-(4-bromophenyl)-1-furan-2-yl-2-methyl-3-oxopentyl]-carbamic acid tert-butylester

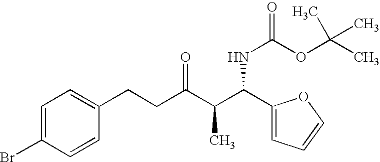

| | | |
|---|---|---|
| Yield: | 164 mg | (94 % of theor.) |
| DC: | R_{f} = 0.24 | (n-pentane/Et₂O:4/1) |
| Melting point: | 117° C. | |
| Excess diastereomer: | de ≧ 96% | (NMR) |
| Excess enantiomer: | ee ≧ 96% | (per de (S, R, S)-37e) |
| Amount of rotation: | [α]_{D}²⁴ = −29.0 | (CHCl₃, c = 0.50) |

¹H-NMR spectrum (400 MHz, (CD₃)₂CO):

δ=1.09 (d, 3H, J=7.1, CHCH₃), 1.38 (s, 9H, OC(CH₃)₃), 2.70-3.00 (kB, 4H), CH₂CH₂), 3.21 (m, 1H, CHCH₃), 4.95 (m, 1H, CHNH), 6.23 (m, 1H, CH^{Ar}—C₄H₃O), 6.32-6.39 (kB, 2H, CH^{Ar}—C₄H₃O, NH), 5.90 (d, 2H, J=8.5, CH^{Ar}—C₆H₄Br), 7.42 (d, 2H, J=8.5, CH^{Ar}—C₆H₄Br), 7.45 (m, 1H, CH^{Ar}—C4H₃0) ppm.

¹³C-NMR spectrum (100 MHz, (CD₃)₂CO):

δ=14.3 (CH₃), 28.5 (OC(CH₃)₃), 28.6 (CH₂C₆H₄Br), 43.5 (CH₂CO), 49.6 (CHCH₃), 51.8 (CHNH), 79.1 (OC(CH₃)₃), 107.2 (CH^{Ar}—C₄H₃O), 110.9 (CH^{Ar}—C₄H₃O), 119.8 (C^{Ar}—C₆H₄Br), 131.1 (CH^{Ar}—C₆H₄Br), 131.9 (CH^{Ar}—C₆H₄Br), 141.6 (CH^{Ar}—C₄H₃O), 142.4 (C^{Ar}—C₆H₄Br), 154.3 (C^{Ar}—C₄H₃O), 155.2 (OC═O), 211.4 (C═O) ppm.

IR spectrum (KBr pressed pellet):

\tilde{ν}=3348 (s), 3124 (w), 3035 (w), 2978 (m), 2963 (m), 2930 (m), 2873 (w), 2286 (w), 1897 (w), 1710 (s), 1685 (s), 1603 (w), 1526 (s), 1489 (m), 1457 (m), 1395 (m), 1369 (m), 1327 (m), 1274 (m), 1241 (m), 1168 (s), 1121 (w), 1102 (w), 1070 (m), 1045 (w), 1011 (m), 952 (w), 935 (w), 909 (w), 883 (w), 863 (m), 826 (m), 804 (m), 768 (w), 731 (m), 708 (w), 683 (w), 647 (m), 598 (w), 559 (w), 516 (m), 467 (w) cm⁻¹.

Mass spectrum (El, 70 eV):

m/z (r.l. %)=436 (2, M⁺), 382 (6), 381 (36), 380 (7), 379 (35), 337 (5), 335 (6), 321 (5), 211 (5), 185 (6), 171 (18), 168 (22), 166 (10), 149 (5), 141 (7), 140 (91), 124 (19), 122 (8), 108 (21), 107 (6), 104 (9), 97 (9), 96 (100), 90 (5), 79 (5), 77 (5), 71 (5), 69 (7), 57 (74).

Elemental analysis (C₂₁H₂₇NO₄Br, 436.342):

| | | | |
|---|---|---|---|
| calc.: | C = 57.81 | H = 6.23 | N = 3.21 |
| found: | C = 57.80 | H = 6.19 | N = 3.14 |

Synthesis of the β-Aminoketones According to Formulas XX, XXa, XXb, XXI, XXIa, XXIb from Precursors I, Ib, II, IIa, IIb with Cleavage of the Protective Groups (Tert- Butyloxycarbonyl, Benzyloxycarbonyl)

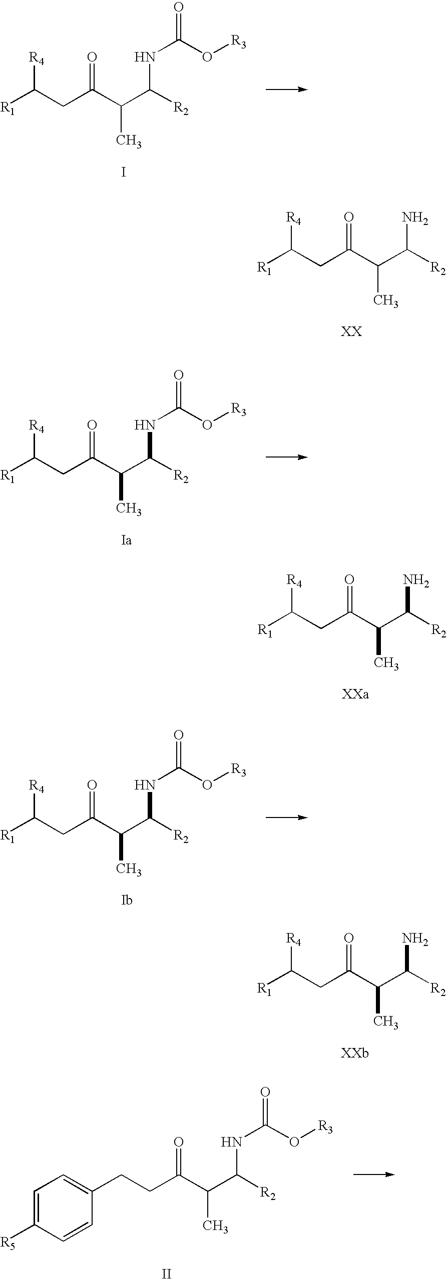

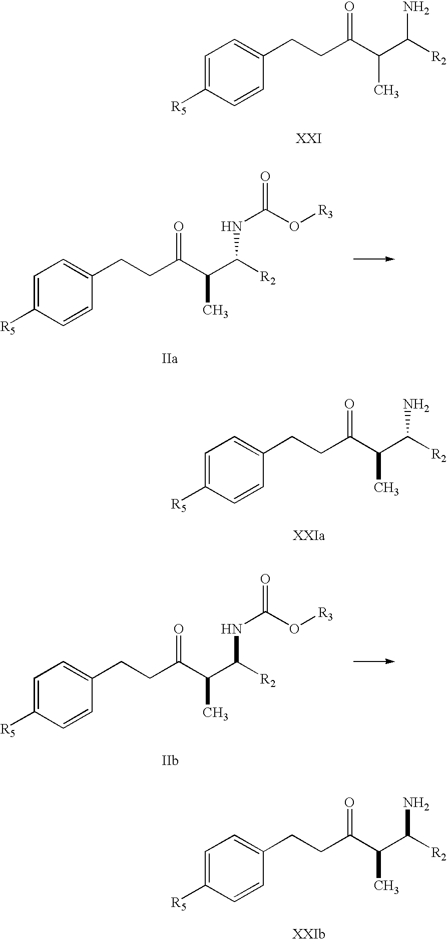

The following processes, which are known from the literature, are possible methods of cleavage, with the reagents:

Trifluoroacetic acid (CF₃COOH) in CH₂Cl₂; trifluoroacetic acid (CF₃COOH) and thiophenol (PhSH); HCl in methanol or ethyl acetate; aqueous HCl in THF or CH₂Cl₂; trimethylsilyliodide (Me₃Sil) in CHCl3 or CH₃CN; aluminium chloride (AlCl₃), methylphenylether (PhOMe), CH₂Cl₂, CH₃NO₂; bromocatecholborane; trimethylsilyl chloride (Me₃SiCl), methylphenylether, CH₂Cl₂; trifluoromethanesulfonic acid (CF₃SO₃H); trimethylsilyltriflate (TMSOTf), PhSMe, trifluoroacetic acid (CF₃COOH); 10% or conc. sulfuric acid in dioxan; tert-butyldimethylsilyltriflate (TBDMSOTf); methanesulfonic acid (Me₃SO₃H) in dioxan or CH₂Cl₂; boron trifluoride etherate (BF₃xEt₂O) in acetic acid (CH₃COOH) or CH₂Cl₂; HF in anisole or pyridine; 33% HBr/glacial acetic acid; BH₃ x THF; hydrogenation H₂, Pd/C; triethylsilane (Et₃SiH), triethylamine Et₃N and PdCl₂ (catalytic); boron tribromide (BBr₃) or boron trichloride (BCl₃) in CH₂Cl₂; sodium/ammonia; bariumhydroxide (BaOH)₂, glyme, water; trifluoroacetic acid (CF₃COOH) and dimethydisulfide (Me₂S) in CH₂Cl₂; 40% KOH, methanol, water; lithiumaluminiumhydride (LiAlH₄), lithiumboron hydride (LIBH₄) or sodium boron hydride (NaBH), trimethylsilylchloride (Me₃SiCl), THF; trifluoroacetic acid (CF₃COOH), bromotrimethylsilane BrSiMe₃ and thioanisole;

All of the compounds of the invention may optionally be present in the form of their racemates, their pure stereoisomers, in particular enantiomers or diastereomers, or in the form of mixtures of the stereoisomers, in particular the enantiomers or diastereomers, in any mixing ratio; in the form illustrated in their respective formulas or in the form of their acids or their bases or in the form of their salts, in particular the physiologically acceptable salts, or in the form of their solvates, in particular the hydrates.

For completeness of disclosure, the following documents evidencing the knowledge and skill of the art are specifically noted:
1) Greene, T. W.; Wuts, P. G. M. Protective Groups in Organic Synthesis; John Wiley & Sons, Inc.; Second Edition; 1991, p. 327-330, 335-338, and literature cited therein.
2) Kocienski, P. J.; Protecting Groups; Georg Thieme Verlag Stuttgart; 1994, p. 192-199, and literature cited therein.
3) Schunk S.; Dissertation, RWTH Aachen, 2002, and literature cited therein.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A beta-amino ketone compound corresponding to formula I 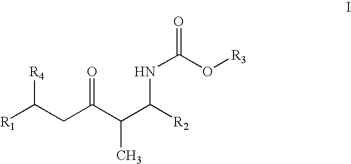
wherein
R₁ is selected from substituted or unsubstituted, branched or unbranched, saturated or unsaturated C₁₋₄ alkyl; or singly or multiply substituted or unsubstituted phenyl;
R₂ is selected from H; substituted or unsubstituted, branched or unbranched, saturated or unsaturated C₁₋₄ alkyl; respectively singly or multiply substituted or unsubstituted aryl or heteroaryl bound by a (CH₂)ₙ group (wherein n=0, 1 or 2);
R₃ is selected from substituted or unsubstituted, branched or unbranched, saturated or unsaturated C₁₋₄ alkyl; or singly or multiply substituted or unsubstituted benzyl; and
R₄ is selected from H; or substituted or unsubstituted, saturated or unsaturated C₁₋₂ alkyl;
or a physiologically acceptable salt or solvate thereof.

2. A compound according to claim 1, wherein said compound is present in the form of a pure enantiomer or diastereoisomer.

3. A compound according to claim 1, wherein said compound is present in the form of a racemic mixture.

4. A compound according to claim 1, wherein said compound is present in the form of a mixture of enantiomers or diastereomers in any mixing ratio.

5. A compound according to claim 1, wherein said compound is present in a stereoselectively pure form having the anti-conformation of formula Ia or the syn-conformation of formula Ib

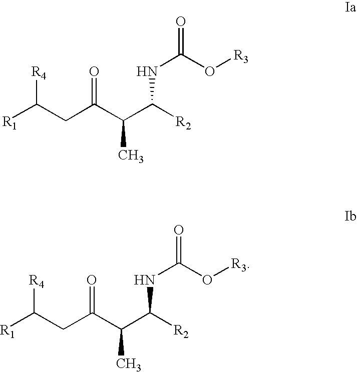

6. A compound according to claim 1, wherein R₁ is selected from the group consisting of methyl, ethyl, iso-propyl and phenyl substituted by R₅ in the para position.

7. A compound according to claim 6, wherein R₅ is selected from the group consisting of OC₁₋₄ alkyl, C₁₋₄ alkyl and halogen.

8. A compound according to claim 1, wherein R₄ is H or CH₃.

9. A compound according to claim 1, wherein R₂ is unsubstituted or singly or multiply substituted aryl or heteroaryl.

10. A compound according to claim 9, wherein R₂ is unsubstituted or singly or multiply substituted phenyl, furyl, thiophenyl or pyridyl.

11. A compound according to claim 10, wherein R₂ is unsubstituted phenyl, furyl, thiophenyl or pyridyl, or R₂ is phenyl substituted by R₆ in the para-position, wherein R₆ is selected from the group consisting of OC₁₋₄ alkyl, C₁₋₄ alkyl and halogen.

12. A compound according to claim 11, wherein R₂ is phenyl substituted by R₆ in the para-position, and R₆ is methoxy, ethoxy, OCF₃, methyl, ethyl, tert.-butyl, i-propyl, CF₃, F, Cl, Br or I.

13. A compound according to claim 1, wherein R₂ is H; substituted or unsubstituted, branched or unbranched, saturated or unsaturated C₃₋₄alkyl; or unsubstituted or singly or multiply substituted benzyl, phenyl, pyridyl, thiophenyl or furyl.

14. A compound according to claim 13, wherein R₂ is H; unsubstituted iso-propyl; unsubstituted phenyl, furyl, thiophenyl or pyridyl; or phenyl substituted by R₆ in the para-position, wherein R₆ is OC₁₋₄ alkyl, C₁₋₄ alkyl or halogen.

15. A compound according to claim 14, wherein R₂ is phenyl substituted by R₆ in the para-position, and R₆ is methoxy, ethoxy, OCF₃, methyl, ethyl, tert.-butyl, i-propyl, CF₃, F, Cl, Br or I.

16. A compound according to claim 1, wherein R₃ is C₂H₅, CH₃, i-propyl, tert.-propyl; or unsubstituted or singly or multiply substituted benzyl.

17. A compound according to claim 16, wherein R₃ is tert.-butyl or unsubstituted benzyl.

18. A compound corresponding to formula XX 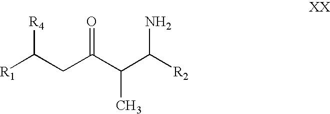
wherein
R₁ is selected from substituted or unsubstituted, branched or unbranched, saturated or unsaturated C₁₋₄ alkyl; or singly or multiply substituted or unsubstituted phenyl;
R₂ is selected from H; substituted or unsubstituted, branched or unbranched, saturated or unsaturated C₁₋₄ alkyl; respectively singly or multiply substituted or unsubstituted aryl or heteroaryl bound by a (CH₂)ₙ group (wherein n=0, 1 or 2); and
R₄ is selected from H; or substituted or unsubstituted, saturated or unsaturated C₁₋₂ alkyl;
or a physiologically acceptable salt or hydrate thereof.

19. A compound according to claim 18, wherein said compound is present in the form of a pure enantiomer or diastereoisomer.

20. A compound according to claim 18, wherein said compound is present in the form of a racemic mixture.

21. A compound according to claim 18, wherein said compound is present in the form of a mixture of enantiomers or diastereomers in any mixing ratio.

22. A compound according to claim 18, wherein said compound is present in a stereoselectively pure form having the anti-conformation according to formula XXa or the syn-conformation according to formula XXb

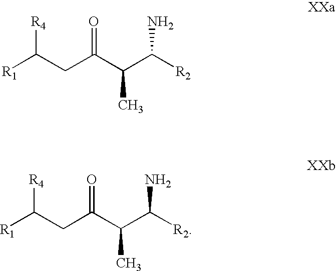

23. A compound according to claim 18, wherein R₁ is selected from the group consisting of methyl, ethyl, iso-propyl and phenyl substituted by R₅ in the para position.

24. A compound according to claim 23, wherein R₅ is selected from the group consisting of OC₁₋₄ alkyl, C₁₋₄ alkyl and halogen.

25. A compound according to claim 24, wherein R₅ is selected from the group consisting of fluorine, chlorine, bromine, iodine, methyl, ethyl, iso-propyl, iso-butyl, tert.-butyl, methoxy and ethoxy.

26. A compound according to claim 25, wherein R₅ is bromine, iodine, tert.-butyl or methoxy.

27. A compound according to claim 18, wherein R₄ is H or CH₃.

28. A compound according to claim 18, wherein R₂ is unsubstituted or singly or multiply substituted aryl or heteroaryl.

29. A compound according to claim 28, wherein R₂ is unsubstituted or singly or multiply substituted phenyl, furyl, thiophenyl or pyridyl.

30. A compound according to claim 29, wherein R₂ is unsubstituted phenyl, furyl, thiophenyl or pyridyl, or R₂ is phenyl substituted by R₆ in the para-position, wherein R₆ is selected from the group consisting of OC₁₋₄ alkyl, C₁₋₄ alkyl and halogen.

31. A compound according to claim 30, wherein R₂ is phenyl substituted by R₆ in the para-position, and R₆ is methoxy, ethoxy, OCF₃, methyl, ethyl, tert.-butyl, i-propyl, CF₃, F, Cl, Br or I.

32. A compound according to claim 18, wherein R₂ is H; substituted or unsubstituted, branched or unbranched, saturated or unsaturated C₃₋₄alkyl; or unsubstituted or singly or multiply substituted benzyl, phenyl, pyridyl, thiophenyl or furyl.

33. A compound according to claim 32, wherein R₂ is H; unsubstituted iso-propyl; unsubstituted phenyl, furyl, thiophenyl or pyridyl; or phenyl substituted by R₆ in the para-position, wherein R₆ is OC₁₋₄ alkyl, C₁₋₄ alkyl or halogen.

34. A compound according to claim 33, wherein R₂ is phenyl substituted by R₆ in the para-position, wherein R₆ is methoxy, ethoxy, OCF₃, methyl, ethyl, tert.-butyl, i-propyl, CF₃, F, Cl, Br or I.

35. A compound corresponding to formula II 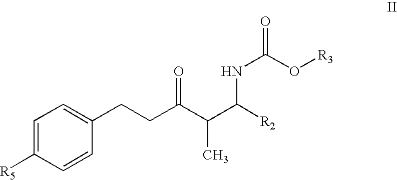
wherein
R₂ is H; substituted or unsubstituted, branched or unbranched, saturated or unsaturated C₁₋₄ alkyl; or respectively singly or multiply substituted or unsubstituted aryl or heteroaryl bound by a (CH₂)ₙ group (wherein n=0, 1 or 2);
R₃ is substituted or unsubstituted, branched or unbranched, saturated or unsaturated C₁₋₄ alkyl; or singly or multiply substituted or unsubstituted benzyl; and
R₅ is OC₁₋₄ alkyl or C₁₋₄ alkyl, respectively substituted or unsubstituted, branched or unbranched, saturated or unsaturated, or halogen;
or a physiologically acceptable salt or solvate thereof.

36. A compound according to claim 35, wherein said compound is present in a stereoselectively pure form having the anti-conformation according to formula IIa or the syn-conformation according to formula IIb

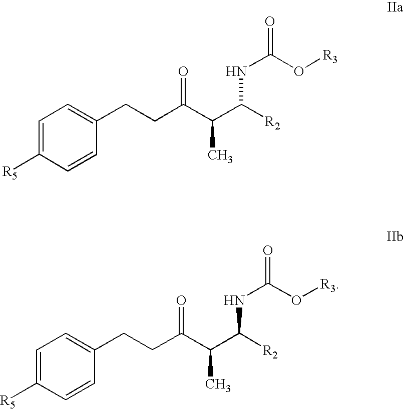

37. A compound according to claim 35, wherein R₅ is selected from the group consisting of fluorine, chlorine, bromine, iodine, methyl, ethyl, iso-propyl, iso-butyl, tert.-butyl, methoxy and ethoxy.

38. A compound according to claim 37, wherein R₅ is bromine, iodine, tert.-butyl or methoxy.

39. A compound according to claim 35, wherein R₂ is unsubstituted or singly or multiply substituted aryl or heteroaryl.

40. A compound according to claim 39, wherein R₂ is unsubstituted or singly or multiply substituted phenyl, furyl, thiophenyl or pyridyl.

41. A compound according to claim 40, wherein R₂ is unsubstituted phenyl, furyl, thiophenyl or pyridyl, or R₂ is phenyl substituted by R₆ in the para-position, wherein R₆ is selected from the group consisting of OC₁₋₄ alkyl, C₁₋₄ alkyl and halogen.

42. A compound according to claim 41, wherein R₂ is phenyl substituted by R₆ in the para-position, and R₆ is methoxy, ethoxy, OCF₃, methyl, ethyl, tert.-butyl, i-propyl, CF₃, F, Cl, Br or I.

43. A compound according to claim 35, wherein R₂ is H; substituted or unsubstituted, branched or unbranched, saturated or unsaturated C₃₋₄alkyl; or unsubstituted or singly or multiply substituted benzyl, phenyl, pyridyl, thiophenyl or furyl.

44. A compound according to claim 43, wherein R₂ is H; unsubstituted iso-propyl; unsubstituted phenyl, furyl, thiophenyl or pyridyl; or phenyl substituted by R₆ in the para-position, wherein R₆ is OC₁₋₄ alkyl, C₁₋₄ alkyl or halogen.

45. A compound according to claim 44, wherein R₂ is phenyl substituted by R₆ in the para-position, and R₆ is methoxy, ethoxy, OCF₃, methyl, ethyl, tert.-butyl, i-propyl, CF₃, F, Cl, Br or I.

46. A compound according to claim 35, wherein R₃ is C₂H₅, CH₃, i-propyl, tert.-propyl; or unsubstituted or singly or multiply substituted benzyl.

47. A compound according to claim 46, wherein R₃ is tert.-butyl or unsubstituted benzyl.

48. A compound corresponding to formula XXI 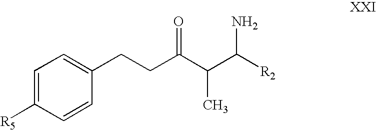
wherein
R₂ is H; substituted or unsubstituted, branched or unbranched, saturated or unsaturated C₁₋₄ alkyl; respectively singly or multiply substituted or unsubstituted aryl or heteroaryl bound by a (CH₂)ₙ group (wherein n=0, 1 or 2); and
R₅ is OC₁₋₄ alkyl or C₁₋₄ alkyl, respectively substituted or unsubstituted, branched or unbranched, saturated or unsaturated, or halogen;
or a physiologically acceptable salt or solvate thereof.

49. A compound corresponding to claim 48, wherein said compound is present in a stereoselectively pure form having the anti-conformation according to formula XXIa or the syn-conformation according to formula XXIb

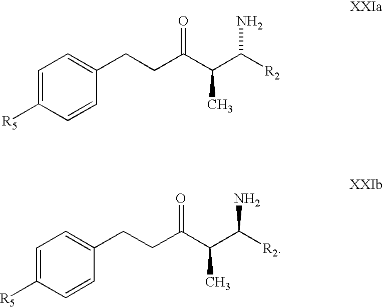

50. A compound according to claim 48, wherein R₅ is selected from the group consisting of fluorine, chlorine, bromine, iodine, methyl, ethyl, iso-propyl, iso-butyl, tert.-butyl, methoxy and ethoxy.

51. A compound according to claim 50, wherein R₅ is bromine, iodine, tert.-butyl or methoxy.

52. A compound according to claim 48, wherein R₂ is unsubstituted or singly or multiply substituted aryl or heteroaryl.

53. A compound according to claim 52, wherein R₂ is unsubstituted or singly or multiply substituted phenyl, furyl, thiophenyl or pyridyl.

54. A compound according to claim 53, wherein R₂ is unsubstituted phenyl, furyl, thiophenyl or pyridyl, or R₂ is phenyl substituted by R₆ in the para-position, wherein R₆ is selected from the group consisting of OC₁₋₄ alkyl, C₁₋₄ alkyl and halogen.

55. A compound according to claim 54, wherein R₂ is phenyl substituted by R₆ in the para-position, and R₆ is methoxy, ethoxy, OCF₃, methyl, ethyl, tert.-butyl, i-propyl, CF₃, F, Cl, Br or I.

56. A compound according to claim 48, wherein R₂ is H; substituted or unsubstituted, branched or unbranched, saturated or unsaturated C₃₋₄alkyl; or unsubstituted or singly or multiply substituted benzyl, phenyl, pyridyl, thiophenyl or furyl.

57. A compound according to claim 56, wherein R₂ is H; unsubstituted iso-propyl; unsubstituted phenyl, furyl, thiophenyl or pyridyl; or phenyl substituted by R₆ in the para-position, wherein R₆ is OC₁₋₄ alkyl, C₁₋₄ alkyl or halogen.

58. A compound according to claim 57, wherein R₂ is phenyl substituted by R₆ in the para-position, and R₆ is methoxy, ethoxy, OCF₃, methyl, ethyl, tert.-butyl, i-propyl, CF₃, F, Cl, Br or I.

59. A compound according to claim 1, selected from the group consisting of:
[5-(4-tert-butyl-phenyl)-1-isopropyl-2-methyl-3-oxo-pentyl]-carbamic acid benzylester,
[1-benzyl-5-(4-bromo-phenyl)-2-methyl-3-oxo-pentyl]-carbamic acid benzylester,
[5-(4-bromo-phenyl)-2-methyl-3-oxo-1-phenyl-pentyl]-carbamic acid tert-butylester,
[5-(4-bromo-phenyl)-1-furan-2-yl-2-methyl-3-oxo-pentyl]-carbamic acid tert-butylester,
[5-(4-bromo-phenyl)-1-(4-tert-butyl-phenyl)-2-methyl-3-oxo-pentyl]-carbamic acid tert-butylester,
[5-(4-bromo-phenyl)-1-(4-methoxy-phenyl)-2-methyl-3-oxo-pentyl]-carbamic acid tert-butylester,
[5-(4-bromo-phenyl)-2-methyl-3-oxo-1-p-tolyl-pentyl]-carbamic acid tert-butylester,
[5-(4-bromo-phenyl)-2-methyl-3-oxo-pentyl]-carbamic acid tert-butylester,
[5-(4-tert-butyl-phenyl)-1-furan-2-yl-2-methyl-3-oxo-pentyl]-carbamic acid tert-butylester,
[5-(4-methoxy-phenyl)-2-methyl-3-oxo-1-phenyl-pentyl]-carbamic acid tert-butylester,
(2-methyl-3-oxo-1-phenyl-heptyl)-carbamic acid tert-butylester
[5-(4-tert-butyl-phenyl)-2-methyl-3-oxo-1-phenyl-pentyl]-carbamic acid tert-butyl ester.
[5-(4-methoxyphenyl)-2-methyl-3-oxo-1-(4-trifluoromethylphenyl)pentyl]-carbamic acid tert-butylester;
or a physiologically acceptable salt or solvate thereof.

60. A compound according to claim 59, wherein said compound is present in the form of a pure enantiomer or diastereoisomer.

61. A compound according to claim 59, wherein said compound is present in the form of a racemic mixture.

62. A compound according to claim 59, wherein said compound is present in the form of a mixture of enantiomers or diastereomers in any mixing ratio.

63. A process for producing a beta-amino ketone compound corresponding to formula I according to claim 1, as shown in reaction diagram III: 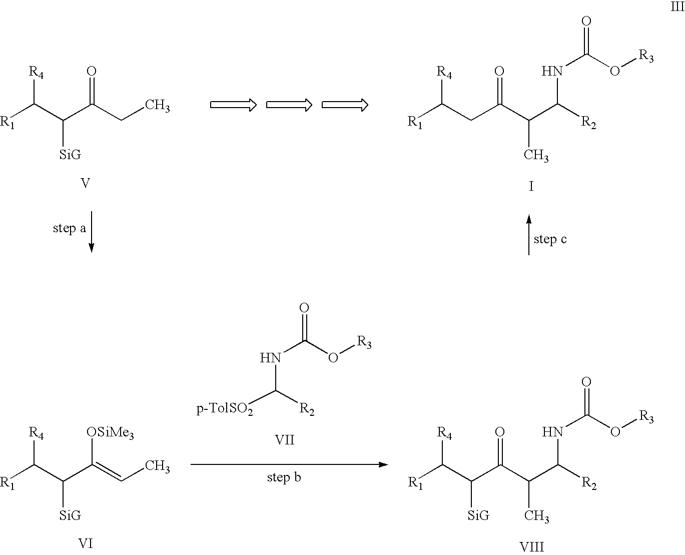
wherein
GSi=tert.-hexyl-(CH₂)₂Si;
and wherein
in step a:
the temperature is kept at <−70° C.,
a non-polar organic solvent is used,
TMSCI is used, and
the reaction takes place in the presence of a base;
in step b:
the temperature is kept at <0° C.,
a polar or non-polar organic solvent is used, and
the reaction takes place in the presence of a Lewis acid; and
in step c:
the temperature is kept at <0° C.,
a non-polar organic solvent is used, and
TBAF or HF is used.

64. A process according to claim 63, wherein
in step a:
the temperature is kept at <−78° C.,
THF is used as the non-polar organic solvent,
LDA is used as the base, and
TMSCI is used.

65. A process according to claim 63, wherein
in step b:
the temperature is kept at <−78° C.,
CH₂Cl₂ is used as the polar organic solvent,
TiCl₄ or SnCl₄ is used as the Lewis acid, and
KH is additionally used.

66. A process according to claim 63, wherein
in step c:
the temperature is kept at <−78° C.,
THF is used as the non-polar organic solvent,
NH₄F is used to adjust a pH <7, and
TBAF or HF is used.

67. A process for producing a beta-amino ketone compound corresponding to formula II according to claim 35, as shown in reaction diagram IIIa: 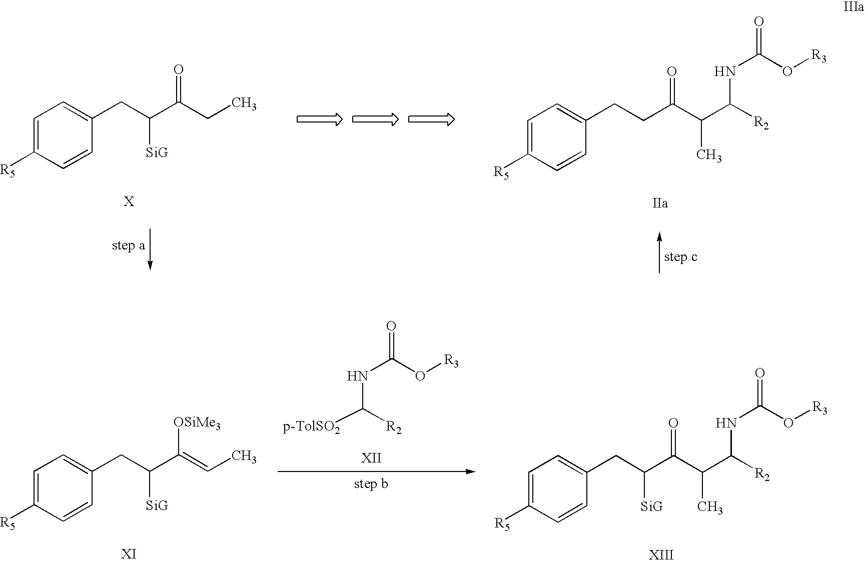
wherein
GSi=tert.-hexyl-(CH₂)₂Si,
and wherein
in step a:
the temperature is kept at <−70° C.,
a non-polar organic solvent is used,
TMSCI is used, and
the reaction takes place in the presence of a base;
in step b:
the temperature is kept at <0° C.,
a polar or non-polar organic solvent is used, and
the reaction takes place in the presence of a Lewis acid; and
in step c:
the temperature is kept at <0° C.,
a non-polar organic solvent is used, and
TBAF or HF is used.

68. A process according to claim 67, wherein
in step a:
the temperature is kept at <−78° C.,
THF is used as the non-polar organic solvent,
LDA is used as the base, and
TMSCI is used.

69. A process according to claim 67, wherein
in step b:
the temperature is kept at <−78° C.,
CH₂Cl₂ is used as the polar organic solvent,
TiCl₄ or SnCl₄ is used as the Lewis acid, and
KH is additionally used.

70. A process according to claim 67, wherein
in step c:
the temperature is kept at <−78° C.,
THF is used as the non-polar organic solvent,
NH₄F is used to adjust a pH <7, and
TBAF or HF is used.

71. A process for stereoselectively producing an anti-beta-amino ketone compound corresponding to formula IIa according to claim 36, as shown in reaction diagram IIIb: 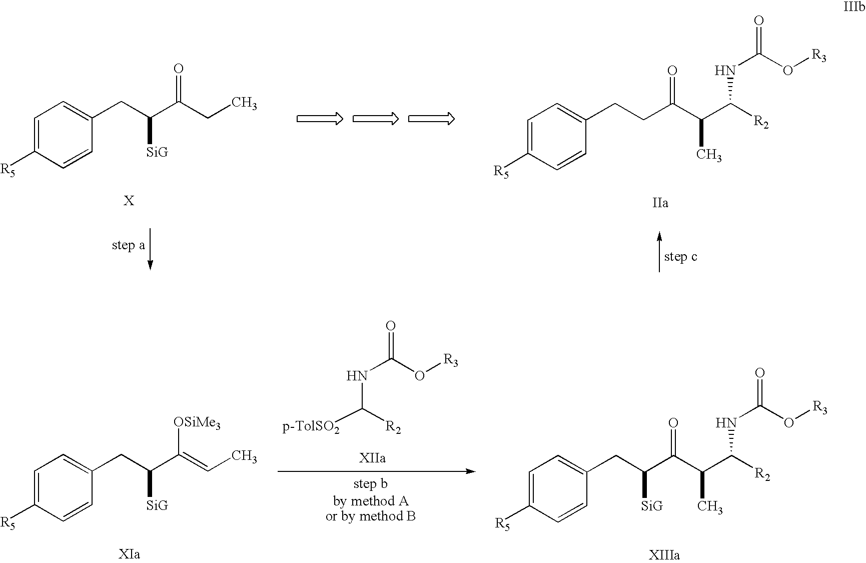
wherein
GSi=tert.-hexyl-(CH₂)₂Si,
and wherein
in step a:
the temperature is kept at <−70° C.,
a non-polar organic solvent is used,
TMSCI and HMPA are used, and
the reaction takes place in the presence of a base;
in step b:
the temperature is kept at <−70° C.,
a polar organic solvent is used,
and the reaction takes place in the presence of a Lewis acid; and
in step c:
the temperature is kept at <−70° C.,
a non-polar organic solvent is used,
TBAF or HF is used, and
the reaction takes place at a pH <7.

72. A process according to claim 71, wherein
in step a:
the temperature is kept at <−78° C., and/or
THF is used as the non-polar organic solvent, and/or
LDA is used as the base; and
TMSCI and HMPA are used.

73. A process according to claim 71, wherein
in step b:
the temperature is kept at <−78° C.,
CH₂Cl₂ is used as the polar organic solvent,
TiCl₄ or SnCl₄ is used as the Lewis acid; and
KH is additionally used.

74. A process according to claim 71, wherein
in step c:
the temperature is kept at <−78° C.,
THF is used as the non-polar organic solvent,
NH₄F is used to adjust a pH <7, and
TBAF or HF is used.

75. A process for producing a beta-amino ketone compound corresponding to formula I according to claim 1, as shown in reaction diagram IV 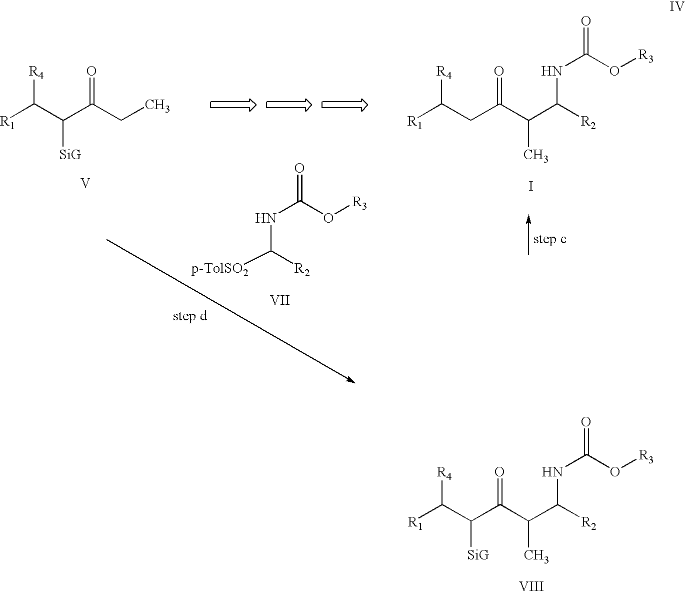
wherein
GSi=tert.-hexyl-(CH₂)₂Si,
and wherein
in step d:
the temperature is kept at <0° C.,
a non-polar organic solvent is used, and
the reaction takes place in the presence of a base; and
in step c:
the temperature is kept at <0° C.,
a non-polar organic solvent is used,
TBAF or HF is used.

76. A process according to claim 75, wherein
in step d:
the temperature is kept at <−78° C.,
THF is used as the non-polar organic solvent,
HMPA is used, and
a lithium base is used as the base.

77. A process according to claim 75, wherein
in step c:
the temperature is kept at <−78° C.,
THF is used as the non-polar organic solvent,
NH₄F is used to adjust a pH <7, and
TBAF or HF is used.

78. A process for stereoselectively producing a syn-beta-amino ketone compound corresponding to formula Ib according to claim 5, as shown in reaction diagram IVa 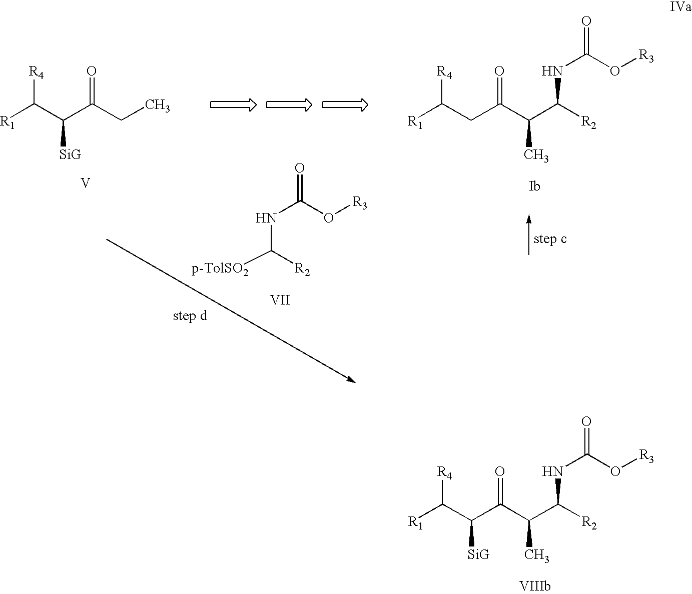
wherein
GSi=tert.-hexyl-(CH₂)₂Si,
and wherein
in step d:
the temperature is kept at <−70° C.,
a non-polar organic solvent is used,
HMPA is used, and
the reaction takes place in the presence of a base; and
in step c:
the temperature is kept at <−70° C.,
a non-polar organic solvent is used,
TBAF or HF is used, and
the reaction takes place at a pH <7.

79. A process according to claim 78, wherein
in step d:
the temperature is kept at <−78° C.,
THF is used as the non-polar organic solvent,
HMPA is used, and
a lithium base is used as the base.

80. A process according to claim 78, wherein
in step c:
the temperature is kept at <−78° C.,
THF is used as the non-polar organic solvent,
NH₄F is used to adjust a pH <7, and
TBAF or HF is used.

81. A process for producing a beta-amino ketone compound corresponding to formula II according to claim 35, as shown in reaction diagram IVb 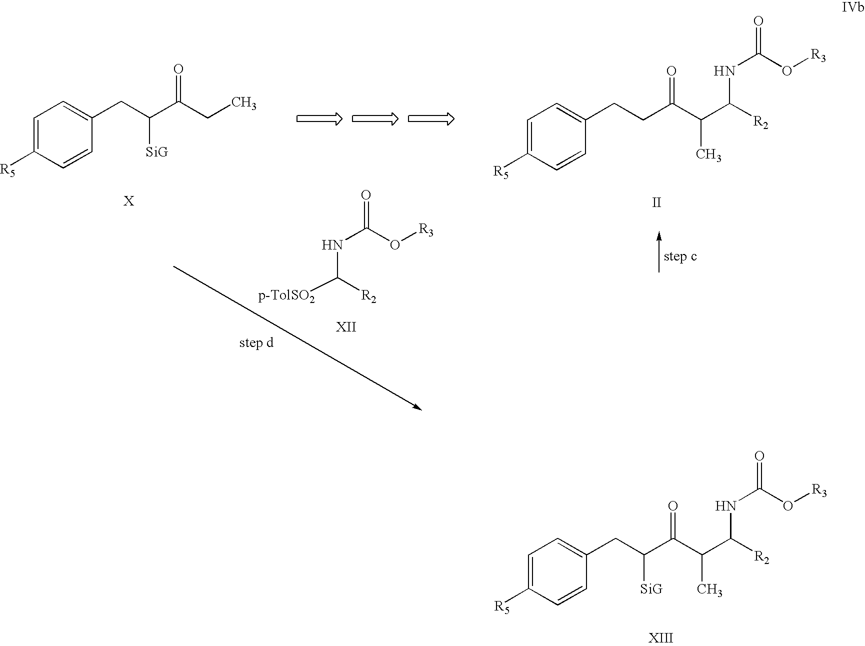
wherein
GSi=tert.-hexyl-(CH₂)₂Si,
and wherein
in step d:
the temperature is kept at <0° C.,
a non-polar organic solvent is used, and
the reaction takes place in the presence of a base; and
in step c:
the temperature is kept at <0° C.,
a non-polar organic solvent is used, and
TBAF or HF is used.

82. A process according to claim 81, wherein
in step d:
the temperature is kept at <−78° C.,
THF is used as the non-polar organic solvent,
HMPA is used, and
a lithium base is used as the base.

83. A process according to claim 81, wherein
in step c:
the temperature is kept at <−78° C.,
THF is used as the non-polar organic solvent,
NH₄F is used to adjust a pH <7, and
TBAF or HF is used.

84. A process for stereoselectively producing an anti-beta-amino ketone compound corresponding to formula IIb according to claim 36, as shown in reaction diagram IVc 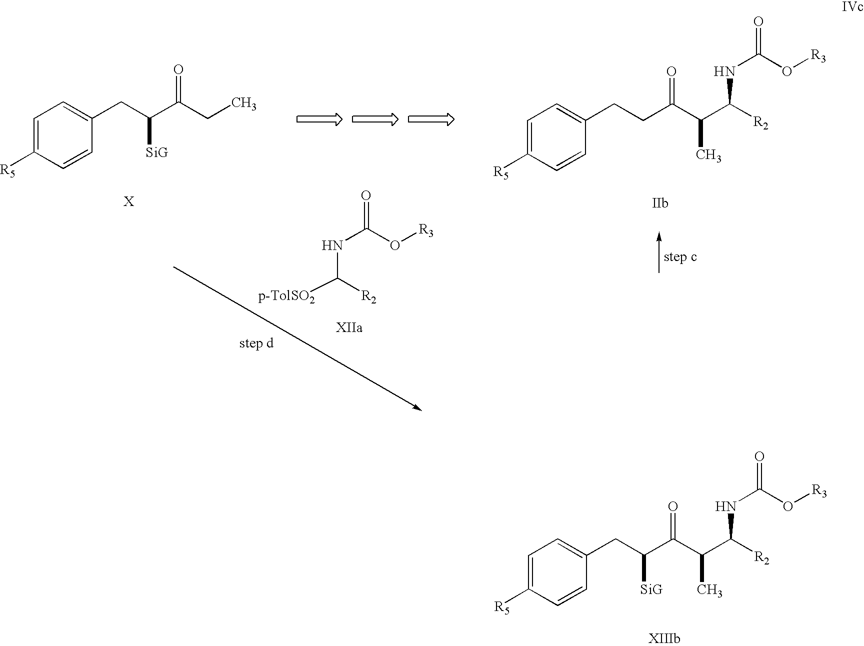
wherein
GSi=tert.-hexyl-(CH₂)₂Si,
and wherein
in step d:
the temperature is <−70° C.,
a non-polar organic solvent is used,
HMPA is used, and
the reaction takes place in the presence of a base; and
in step c:
the temperature is kept at <−70° C.,
a non-polar organic solvent is used,
TBAF or HF is used, and
the reaction takes place at a pH <7.

85. A process according to claim 84, wherein
in step d:
the temperature is kept at <−78° C.,
THF is used as the non-polar organic solvent,
HMPA is used, and
a lithium base is used as the base.

86. A process according to claim 84, wherein
in step c:
the temperature is kept at <−78° C.,
THF is used as the non-polar organic solvent,
NH₄F is used to adjust a pH <7, and
TBAF or HF is used.

87. A process according to claim 63, wherein R₃ is tert.-butyl, further comprising reacting the produced compound with trifluoroacetic acid and removing the solvent, whereby said compound is obtained in the form of a triflate.

88. A process according to claim 63, wherein R₃ is other than tert.-butyl, further comprising reacting the produced compound with HCl and removing the solvent.

89. A process according to claim 75, wherein R₃ is tert.-butyl, further comprising reacting the produced compound with trifluoroacetic acid and removing the solvent, whereby said compound is obtained in the form of a triflate.

90. A process according to claim 75, wherein R₃ is other than tert.-butyl, further comprising reacting the produced compound with HCl and removing the solvent.

91. A pharmaceutical composition comprising at least one compound according to 1, and at least one further ingredient selected from the group consisting of pharmaceutical carriers, pharmaceutically active ingredients, pharmaceutical auxiliaries and pharmaceutical additives.

92. A pharmaceutical composition comprising at least one compound according to 18, and at least one further ingredient selected from the group consisting of pharmaceutical carriers, pharmaceutically active ingredients, pharmaceutical auxiliaries and pharmaceutical additives.

93. A pharmaceutical composition comprising at least one compound according to 35, and at least one further ingredient selected from the group consisting of pharmaceutical carriers, pharmaceutically active ingredients, pharmaceutical auxiliaries and pharmaceutical additives.

94. A pharmaceutical composition comprising at least one compound according to 48, and at least one further ingredient selected from the group consisting of pharmaceutical carriers, pharmaceutically active ingredients, pharmaceutical auxiliaries and pharmaceutical additives.

95. A method of treating a condition selected from the group consisting of pain, anxiety, depression and epilepsy in a patient in need of such treatment, said method comprising administering to said patient a pharmaceutically effective amount of a compound according to claim 1.

96. A method according to claim 95, wherein said condition is pain.

97. A method of treating a condition selected from the group consisting of pain, anxiety, depression and epilepsy in a patient in need of such treatment, said method comprising administering to said patient a pharmaceutically effective amount of a compound according to claim 18.

98. A method according to claim 97, wherein said condition is pain.

99. A method of treating a condition selected from the group consisting of pain, anxiety, depression and epilepsy in a patient in need of such treatment, said method comprising administering to said patient a pharmaceutically effective amount of a compound according to claim 35.

100. A method according to claim 99, wherein said condition is pain.

101. A method of treating a condition selected from the group consisting of pain, anxiety, depression and epilepsy in a patient in need of such treatment, said method comprising administering to said patient a pharmaceutically effective amount of a compound according to claim 48.

102. A method according to claim 101, wherein said condition is pain.
